(19) United States
(12) Patent Application Publication
(10) Pub. No.: US 2015/0272970 A1
(43) Pub. Date: Oct. 1, 2015

(54) TARGETING CHEMOTHERAPY AGENT RESISTANCE IN CANCER

(71) Applicants: Carlos CORDON-CARDO, (US); Josep DOMINGO-DOMENECH, New York, NY (US)

(72) Inventors: Carlos Cordon-Cardo, New York, NY (US); Josep Domingo-Domenech, New York, NY (US)

(21) Appl. No.: 14/426,906

(22) PCT Filed: Sep. 9, 2013
(86) PCT No.: PCT/US13/58805

§ 371 Date: Mar. 9, 2015

Related U.S. Application Data
(60) Provisional application No. 61/698,095, filed on Sep. 7, 2012.

(51) Int. Cl. A61K 31/635; C12Q 1/68; G01N 33/574; A61K 31/5377; A61K 45/06

(52) U.S. Cl. CPC A61K 31/635; A61K 31/5377; A61K 45/06; G01N 33/57496; C12Q 1/6886; G01N 2800/52; C12Q 2600/158

(57) ABSTRACT

The present technology provides methods of identifying tumor cells that are resistant to one or more chemotherapy agents, as well as preventing or reducing such resistance in cancer, and predicting survivability of a subject having cancer.

### BACKGROUND

The present technology relates generally to methods of identifying tumor cells that are resistant to chemotherapy agents, preventing or reducing resistance to such agents in cancer (such as prostate cancer), and predicting survivability of a subject having cancer.

Prostate cancer is the most common cancer diagnosis and second leading cause of cancer related death in men (Jemal et al., 2011). Despite the availability of local treatment, many patients relapse after primary therapy. Initially, relapsed prostate cancer patients have a hormone-dependent disease that responds to androgen withdrawal. However, despite hormonal manipulations, prostate cancer often progresses to a hormone refractory state (Pound et al., 1999).

Acquisition of chemotherapy resistance is a devastating and widespread phenomenon in clinical oncology, including in cancers such as prostate cancer. Docetaxel is one example of a taxane anti-mitotic agent, and is currently used as the standard therapy for patients with hormone-refractory prostate cancer (HRPC) (Petrylak et al., 2004; Tannock et al., 2004). Docetaxel was the first chemotherapy agent shown to improve survival of HRPC patients. Unfortunately, patients who initially respond eventually acquire resistance, and this event precedes therapeutic stalemate and death, as limited effective therapies currently exist in this context. Presently, the main identified mechanisms of acquired resistance relate to the expression of β-tubulin isoforms/mutations and the activation of drug efflux pumps, among others (Mahon et al., 2011; Seruga et al., 2011). Unfortunately, in spite of these advances, treatment of patients who become resistant to chemotherapy agents such as Docetaxel remains a critical clinical challenge. Therapeutic strategies that target Docetaxel resistant cells remain elusive. There exists a strong need for a new therapeutic strategy to identify and effectively target tumor cells that are resistant to chemotherapy agents. Such a strategy would allow for the prediction of cancer survivability and would provide a method for preventing and reducing resistance in cancers such as prostate cancer, to chemotherapy agents such as Docetaxel.

### BRIEF SUMMARY

In certain embodiments, the present technology is directed to a method of preventing or reducing resistance to a first chemotherapy agent in a cancer, the method comprising administering to a subject having the cancer one or both of a Notch signaling pathway inhibitor or a Hedgehog signaling pathway inhibitor.

In certain embodiments, the methods further comprise administering a further chemotherapy agent to the subject, the further chemotherapy agent being the same or different from the first chemotherapy agent. The further chemotherapy agent can be administered prior to, after, or concurrently with, the Notch signaling pathway inhibitor or Hedgehog signaling pathway inhibitor. In certain embodiments, the Notch signaling pathway inhibitor is a Notch antibody, a nucleic acid that inhibits Notch activity (for example, a short hairpin RNA or a nucleic acid that is complementary to a Notch nucleic acid or fragment thereof), DBZ, Compound E, or a PI3K/AKT pathway inhibitor (for example, LY294002).

In certain embodiments, the Hedgehog signaling pathway inhibitor is a Hedgehog antibody, a nucleic acid that inhibits Hedgehog activity Cyclopamine, GDC-0449, a Bcl-2 family member inhibitor (for example, ABT-737), or a short hairpin RNA that targets Gli1 or Gli2.

In other embodiments, the present technology is directed to a method of treating cancer, the method comprising administering to a subject having the cancer one or both of a Notch signaling pathway inhibitor or a Hedgehog signaling pathway inhibitor.

In other embodiments, the present technology is directed to a method of identifying a tumor cell that is resistant to a chemotherapy agent, the method comprising detecting activation of the Notch or Hedgehog signaling pathways, wherein the activation indicates the resistant tumor cell.

In other embodiments, the present technology is directed to a method of predicting the predicting survival of a subject having cancer, the method comprising detecting activation of the Notch or Hedgehog signaling pathways, wherein said activation indicates a decreased survival time.

In certain embodiments, the further activation indicates tumor aggressiveness and poor patient prognosis. In certain embodiments, the subject has previously received treatment for the cancer, for example, comprising administration of a chemotherapy agent, including but not limited to Docetaxel.

In certain embodiments, the detecting activation comprises detection of one or more of:

a) cleaved Notch2;

b) increased expression of Gli1;

c) increased expression of Gli2;

d) reduced expression of Patched;

d) phosphorylation of AKT (Ser473); or

e) increased levels of Bcl-2.

In other embodiments, the present technology is directed to a method of identifying a tumor cell resistant to a chemotherapy agent, the method comprising detecting decreased expression of an HLA class I antigens, a cytokeratin 18, a cytokeratins 19 or any combination thereof compared to a normal control cell, wherein the decreased expression indicates a tumor cell that is resistant to the chemotherapy agent (including but not limited to Docetaxel.

In certain embodiments, the present technology is directed to methods of preventing or reducing resistance to one or more chemotherapy agents in cancer by administering to a subject suffering from the cancer a Notch signaling pathway inhibitor, a Hedgehog signaling pathway inhibitor or both. In certain embodiments, the subject is administered a chemotherapy agent such as, but not limited to, Docetaxel. In such embodiments, the chemotherapy agent is administered prior to, after or concurrently with the Notch inhibitor and/or Hedgehog inhibitor.

In other embodiments, the present technology provides methods of treating a tumor that is resistant to one or more chemotherapy agents by administering to a subject in need thereof a Notch signaling pathway inhibitor, a Hedgehog signaling pathway inhibitor or both.

In other embodiments, the present technology is directed to methods of identifying a tumor cell that is resistant to one or more chemotherapy agents, by detecting activation of the Notch or Hedgehog signaling pathways, downregulation of HLA class I antigens, downregulation of cytokeratin 18, cytokeratin 19 or any combination thereof. In certain embodiments, activation or the Notch or Hedgehog signaling pathways or downregulation of indicates a tumor cell that is resistant to one or more chemotherapy agents such as, but not limited to, Docetaxel.

In other embodiments, the present technology provides a method of predicting survival or of subjects having cancer such as, but not limited to prostate cancer, by detecting activation of the Notch or Hedgehog signaling pathways, downregulation of HLA class I antigens, downregulation of cytokeratin 18 cytokeratin 19 or any combination thereof. Activation or the Notch or Hedgehog signaling pathways or downregulation of indicates a decrease survival time, poor prognosis or tumor aggressiveness. As used herein, “survival” means overall survival or recurrence-free survival time.

Notch signaling pathways inhibitors include, for example, a Notch antibody, a nucleic acid that inhibits Notch expression or activity, DBZ [(2S)-2-[2-(3,5-difluorophenyl)-acetylamino]-N-(5-methyl-6-oxo-6,7-dihydro-5H-dibenzo[b,-d]azepin-7-yl)-propionamide], Compound E (CAS 209986-17-4), or PI3K/AKT pathway inhibitors, such as LY294002 [2-(4-Morpholinyl)-8-phenyl-1(4H)-benzopyran-4-one hydrochloride]. A nucleic acid is for example, a short hairpin RNA or a nucleic acid that is complementary to a Notch nucleic acid or fragment thereof. Alternatively, the Notch signaling pathway inhibitor is a nucleic acid that inhibits the expression or activity of one or more molecules in the Notch signaling pathway.

Hedgehog signaling pathway inhibitors include, for example, a Hedgehog antibody, a nucleic acid that inhibits Hedgehog expression or activity Cyclopamine, GDC-0449 (Vismodegib), or Bcl-2 family member inhibitors, such as ABT-737 (CAS 852808-04-9). A nucleic acid is for example, a short hairpin RNA or a nucleic acid that is complementary to a Hedgehog nucleic acid or fragment thereof.

Alternatively, the Hedgehog signaling pathway inhibitor is a nucleic acid that inhibits the expression or activity of one or more molecules in the Hedgehog signaling pathway, for example, Gli1 or Gli2.

Unless otherwise defined, all technical and scientific terms used herein have the same meaning as commonly understood by one of ordinary skill in the art to which the present technology pertains. Although methods and materials similar or equivalent to those described herein can be used in the practice of the present technology, suitable exemplary methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the present technology will be apparent from and encompassed by the following disclosure and claims.

### BRIEF DESCRIPTION OF THE DRAWINGS

FIGS. 1A-E: Characterization of Docetaxel acquired resistance in hormone independent prostate cancer cells. FIG. 1A shows Cell viability assays (MTs) of parental (22Rv1 and DU145) and Docetaxel acquired resistant cells (22Rv1-DR and DU145-DR) treated with increasing doses of Docetaxel during 72 hours. The horizontal gray line specifies the IC50 concentration of the drug (Docetaxel) for sensitive and resistant cells. FIG. 1B shows Colony formation assay of cells treated continuously for 21 days with Docetaxel (25 nM in 22Rv1-DR and 5 nM in DU145-DR cells). The histogram represents the quantitative analysis of the number of colonies formed in parental and Docetaxel resistant cells treated with increasing doses of Docetaxel for 24 hours. FIG. 1C shows Annexin V and propidium iodide staining in sensitive and Docetaxel resistant cells treated with the same concentration of Docetaxel as in FIG. 1B for 72 hours. FIG. 1D shows Immunoblots for Caspase-3 and PARP from the same cells treated with the same conditions as in FIG. 1C. FIG. 1E shows RT-PCR analysis of hematopoietic (CD34), mesenchyme (CDH2) and endothelial (CDH2) lineage markers. Data is represented as mean±SD of three independent experiments.

FIGS. 2A-E: Phenotypical characterization of Docetaxel resistant cells. FIG. 2A shows Genes with at least a 1.8 fold increase (↑) or decrease (↓) in transcript expression comparing parental and Docetaxel resistant (DR) cells. FIG. 2B shows Gene ontology (GO) categories of overlapping genes. Categories with statistical significance (p≦0.01) are represented. GO categories with one asterisk (*) are related to cell proliferation, cell death, and response to drugs. GO categories with two asterisks (**) related to developmental processes. GO categories with three asterisks (***) are related to antigen presentation. FIG. 2C shows cDNA microarray analysis of 22RV1, 22RV1-DR, DU-145, and DU-145-DR gene expression profiles. FIG. 2D shows Immunoblotting and quantification of parental and Docetaxel resistant cells for luminal prostate epithelial differentiation markers, prostate specific markers, basal prostate epithelial differentiation markers, MHC class I antigens, Notch pathway proteins, and Hedgehog pathway proteins. SCaBER was a positive control for high molecular weight cytokeratins and p63. FIG. 2E shows Immunofluorescence of parental and Docetaxel resistant cells shows expression and subcellular localization of cytokeratins (CKs), transcription factors, and HLA class I antigen. Bars correspond to 10 μm.

FIG. 3A-E: Docetaxel resistant cells identified in prostate cancer tissue samples and the association between Docetaxel resistant cells and tumor aggressiveness. FIG. 3A shows Hematoxylin & Eosin (H&E) and immunofluorescence based co-expression analysis in prostate tumors of CKs (CK18+19), HLA class I, and transcription factors with box plot quantifications. White arrows point to CK-negative cells. FIG. 3B shows H&E and immunofluorescence based co-expression analysis in prostate tumors of AR with box plot quantifications. White arrows point to CK-negative cells. FIG. 3C shows CK18 and CK19 immunohistochemistry with and without Docetaxel. The percentage in the upper right hand corner of the boxes indicates the percentage of CK-negative cells. Clinical metastatic prostate cancer tissues treated with Docetaxel have a higher percentage of CK-negative cells than non-treated tissues. FIG. 3D shows Association between the percentage of CK-negative cells and Gleason Score and pathological stage in primary prostate cancer tissues. FIG. 3E shows Kaplan-Meyer analysis of biochemical recurrence-free survival (BRFS) of primary prostate cancer patients (n=31) with low CK-negative content (≦1.3%) compared to high CK-negative content (>1.3%). The two slides depict representative samples with low and high percentages of CK-negative cells. Black arrows point to CK-negative cells. Bars correspond to 100 μm. Data is represented as means±SD.

FIG. 4A-C: Stainings of low molecular weight CK-negative cells in human metastatic prostate tissue samples. CK-negative cells in human metastatic prostate tissue samples do not display a basal phenotype and are located in viable tumor areas. FIG. 4A shows Basal phenotype was analyzed by double-based immunofluorescence staining of low molecular weight CKs (CK18 and CK19) with high molecular weight CKs (CK5 and CK14) and p63 in prostate cancer tissue samples. Left panels illustrate normal prostate tissue used as a control. Magnification microphotographs in the right panels of the metastatic tissue samples show that the identified low molecular weight CK (CK18 and CK19)-negative tumor cells do not express basal markers CK5, CK14, and p63. White arrows point to CK-negative cells with negative basal markers. This analysis was performed in all primary (n=31) and metastatic (n=36) prostate cancer tissue samples and revealed that CK-negative cells do not express basal markers. FIG. 4B shows Representative microphotograph from a bone metastasis illustrated by H&E and cytokeratin staining that CK-negative cells are not in areas of necrosis. Note that cells in necrotic areas are characterized by loss of nuclear structure but still maintain cytokeratin cytoplasmic expression, whereas tumor cells without morphological abnormalities lacking cytokeratin expression are identified in viable tumor areas. The black arrow in the viable tumor panel points to a CK-negative cell in a viable tumor area. This analysis was performed in all primary (n=31) and metastatic (n=36) prostate cancer tissue samples and revealed that CK-negative cells are not located in areas of necrosis. FIG. 4C shows Proliferation status of CK-negative cells was analyzed by double-based immunofluorescence staining of low molecular weight CKs (CK18 and CK19) with Ki67 in metastatic prostate cancer tissue samples. Microphotographs illustrate a CK-negative cell with Ki67 nuclear expression. The histogram summarizes the percentage of CK-negative cells with Ki67-positive nuclear expression. The percentage of CK-negative cells displaying Ki67 staining in metastatic prostate cancer samples was assessed in whole tissue sections by counting the number of tumor cells that lacked cytokeratins (CK18 and CK19) and displayed nuclear expression of Ki67 in 10 contiguous high power fields in three different areas of the tumor. Data is represented as means±SD. Bar corresponds to 100 μm.

FIG. 5: Table of clinic-pathological characteristics and the percentages of CK-negative cells in primary and metastatic prostate cancer patients. The table, related to FIG. 3, summarizes the clinico-pathological characteristics of the 36 metastatic and 31 primary prostate cancer patients from whom the percentage of CK-negative cells was analyzed. Established prognostic factors, such as pre-surgical PSA, tumor differentiation (Gleason score), and extension of the disease (Stage), as well as metastatic site and Docetaxel administration, are represented.

FIG. 6A-G. Docetaxel exposure selects for pre-existing resistant prostate cancer cells. FIG. 6A shows Schematic of two scenarios being tested: transition versus enrichment-selection induced by Docetaxel. FIG. 6B shows Immunofluorescence and flow cytometry quantification of DU145 and 22Rv1 cells showing a population of CK18- and CK19-negative cells. White arrows point to cells with a CK-negative phenotype. FIG. 6C shows Flow cytometry analysis of DU145-pCK19-GFP and 22Rv1-pCK19-GFP cells treated with Docetaxel for 72 hrs. FIG. 6D shows Colony formation assay and quantification of sorted DU145-pCK19-GFP and 22Rv1-pCK19-GFP cells cultured with Docetaxel (10 nM and 50 nM, respectively), or with DMSO, for 72 hours. FIG. 6E shows Time lapse microscopy of DU145-pCK19-GFP cells treated with Docetaxel. Serial images show that a CK19-negative/GFP-negative cell divides in presence of chemotherapy (dotted area), while GFP expressing cells start dying after mitotic arrest. FIG. 6F shows Immunoblots of GFP and Docetaxel-resistance markers in DU145-pCK19-GFP and 22Rv1-pCK19-GFP sorted cells, as well as in unsorted DU145-pCK19-GFP and 22Rv1-pCK19-GFP cells exposed to Docetaxel (72 hours) at the same concentrations as in FIG. 6D. FIG. 6G shows Colony formation assays and quantification of DU145-pCK19-GFP and 22Rv1-pCK19-GFP sorted cells cultured with or without Mitoxantrone (125 nM and 500 nM, respectively), Cisplatin (5 μM and 2.5 μM, respectively), and Vinorelbine (500 nM and 750 nM, respectively) for 72 hours. Data is represented as means±SD of triplicate experiments. * corresponds to p<0.0001.

FIG. 7A-B: Validation and live imaging of the pCK19-GFP reporter system, related to FIG. 6. FIG. 7A shows a Schematic illustration of the generated CK19 promoter-GFP reporter construct; immunofluorescence staining for GFP and CK19 in DU145 and 22Rv1 parental cells with stable integration of pCK19-GFP construct. The white arrow points to a cell lacking the expression of CK19 and GFP. FIG. 7B shows that 22Rv1-pCK19-GFP cells were treated with 50 nM Docetaxel for 48 hours and filmed by time-lapse microscopy. Serial images of a representative experiment show that a 22Rv1-pCK19-GFP-negative cell is able to divide and survive in the presence of Docetaxel (dotted area), while GFP expressing cells start dying after mitotic arrest. The bar corresponds to 10 μm.

FIG. 8A-F: Validation of knockdown and inhibition of Notch and Hedgehog signaling pathway using RNAi and compounds. The data shows that maintenance of Docetaxel resistant prostate cancer cells is dependent on Notch and Hedgehog signaling. FIG. 8A shows Immunoblots that illustrate the decrease in protein expression of cleaved Notch2, Gli1, and Gli2 induced by short hairpin RNAs (shRNAs) against NOTCH2 (#13 and #14), GLI1 (#2 and #5), or GLI2 (#12 and #10) in DU145-pCK19-GFP and 22Rv1-pCK19-GFP cells. FIG. 8B shows Histograms that illustrate the decrease in mRNA expression levels of Notch (NOTCH2, HES1 and HEY1) and Hedgehog (SMO, GLI1 and GLI2) pathway target genes induced by shRNAs. FIG. 8C shows Colony formation assay and quantification of three independent experiments of DU145-pCK19-GFP and 22Rv1-pCK19-GFP sorted cells expressing shRNAs against GLI1 (#2), GLI2 (#12), and NOTCH2 (#13) alone, as well as double and triple combinations. FIG. 8D shows Histograms that illustrate the mRNA expression levels of Notch and Hedgehog pathway target genes when treating DU145-pCK19-GFP and 22RV1-pCK19-GFP cells with Notch inhibitors (DBZ at 1 μM and Compound E (CE) at 1 μM) and Hedgehog inhibitors (Cyclopamine at 1 μM and GDC-0449 at 1 μM) for 72 hours. Results are displayed relative to vehicle (DMSO) exposed controls. FIG. 8E shows how Apoptotic response was analyzed in 22Rv1-pCK19-GFP sorted cells treated for 48 hours with Cyclopamine (1 μM) and/or DBZ (1 μM) by assessing Caspase-3 and PARP cleavage by immunoblotting. FIG. 8F shows Colony formation assay and quantification of colonies derived from 22Rv1-pCK19-GFP sorted cells exposed during 72 hours to Cyclopamine 1 μM (Cyc), GDC-0449 1 μM (GDC), DBZ 1 μM, and Compound-E 1 μM (CE) individually or in combination (Cyc+DBZ or GDC+CE). Data is represented as means±SD of three independent experiments. * corresponds to p<0.05.

FIGS. 9A-F: Dependence of Docetaxel resistant prostate cancer cells on Notch and Hedgehog signaling. FIG. 9A shows Colony formation assay and quantification of DU145-pCK19-GFP and 22Rv1-pCK19-GFP sorted cells expressing shRNAs against GLI1, GLI2, and NOTCH2 alone, as well as double (GM and GLI2) and triple combinations. FIG. 9B shows Flow cytometry analysis of DU145-pCK19-GFP and 22Rv1-pCK19-GFP treated with Cyclopamine (1 μM) and/or DBZ (1 μM) for 48 hours. FIG. 9C shows Immunoblots of Caspase-3 and PARP in DU145-pCK19-GFP cells treated with the same conditions as in FIG. 9B. FIG. 9D shows Colony formation assay and quantification of DU145-pCK19-GFP sorted cells exposed for 72 hours at the same concentrations as in FIGS. 8D, 9B to Cyclopamine, GDC-0449, DBZ, and Compound-E individually or in combination. FIG. 9E shows Flow cytometry analysis after 48-hour administration of Docetaxel alone or in combination with Cyclopamine (1 μM) and/or DBZ (1 μM). 10 nM and 50 nM Docetaxel was used in DU145-pCK19-GFP and 22Rv1-pCK19-GFP cells, respectively. FIG. 9F shows Colony formation assay and quantification of the colonies derived from parental DU145 and 22Rv1 cells exposed to Docetaxel (10 nM and 50 nM, respectively) alone or in combination with Notch inhibitors (CE at 1 μM or DBZ at 1 μM) and/or Hedgehog inhibitors (Cyclopamine at 1 μM or GDC-0449 at 1 μM). Data is represented as means±SD of triplicate experiments. * corresponds to p<0.05.

FIG. 10A-C: Notch and Hedgehog pathway inhibition abrogates the acquisition of Docetaxel resistance in NOD/SCID mice bearing DU145 and 22Rv1 xenografts. FIG. 10A shows Changes in tumor volume of DU145 and 22RV1 xenografts treated with Dexamethasone (Dex) alone, double combinations of Dex with Cyc, DBZ, or Docetaxel, triple combinations of Dex with two other drugs, and quadruple combinations of Dex with Docetaxel, Cyc, and DBZ. Dose schedules were Dexamethasone (15 mg/kg/ip daily), Docetaxel (10 mg/kg/ip once a week for 3 weeks every 4 weeks), DBZ (10 μM/kg/ip daily for 15 days every 4 weeks) and Cyclopamine (50 μg/kg/sc daily). FIG. 10B shows Quantitative RT-PCR of Notch and Hedgehog target genes in DU145 and 22RV1 xenografts obtained from mice treated with the same drugs and concentrations as in FIG. 10A. Bars represent fold-change in mRNA levels relative to vehicle (control). FIG. 10C shows Microphotographs that illustrate the expression of low molecular weight cytokeratins (CK18 and CK19) in DU145 and 22RV1 prostate cancer tumor xenografts in NOD/SCID mice treated for 4 weeks with the same drugs as in FIG. 10A. Magnifications illustrate CK-negative cells. Percentages indicate percentage of CK-negative cells. The histogram represents the percentage of CK-negative cells detected in DU145 and 22RV1 xenografts for each treatment arm. Four xenografts for each treatment group were analyzed. Data is represented as means±SD. * corresponds to p<0.05. The bar corresponds to 100 μm.

FIG. 11A-D: Tumor growth inhibition induced by the combination of Docetaxel with both Notch and Hedgehog pathway inhibitors is not a result of general drug toxicity. FIG. 11A shows Cell viability assays (MTs) in 22Rv1 and DU145 parental cells treated with increasing doses of Etoposide or Docetaxel for 72 hours. The gray horizontal line specifies the IC50 concentration of Etoposide and Docetaxel. Curves represent the quantitative analysis of at least 3 independent experiments. FIG. 11B shows Toxicity (weight loss) in NOD/SCID mice bearing DU145 and 22RV1 tumor xenografts treated with dexamethasone (15 mg/kg/ip daily) plus Docetaxel (10 mg/kg/ip once a week for 3 weeks every 4 weeks) or Etoposide (10 mg/kg/iv once a week for 3 weeks every 4 weeks) in combination with DBZ (10 μM/kg/ip daily for 15 days every 4 weeks) and Cyclopamine (50 μg/kg/sc daily). No mice needed to be euthanized because of a decrease in >20% of their body weights. Data displayed for Docetaxel-treated mice correspond to mice in which tumor volume fold-changes are displayed in FIG. 10A and tumor weights for Etoposide-treated mice correspond to mice in which tumor volumes are displayed in FIG. 11C. FIG. 11D shows Tumor volume changes of DU145 and 22Rv1 tumor xenografts treated with Etoposide and developmental pathway inhibitors. Data corresponds to two independent experiments in 8 mice bearing 2 tumors in the upper flanks for each treatment group and cell line. Data is represented as means±SD.

FIG. 12A-G: Notch and Hedgehog signaling pathway regulation of survival molecules in Docetaxel resistant cells. FIG. 12A shows Immunoblots that illustrate that DU145-pCK19-GFP and 22Rv1-pCK19-GFP express higher levels of p-AKT (Ser-473) and Bcl2 in CK19-negative/GFP-negative cells when compared to CK19-positive/GFP-positive cells. FIG. 12B shows Immunoblots that show that exposure to the Notch inhibitor, DBZ (1 μM), for 72 hours decreases p-AKT (Ser-473) activity and that exposure to Hedgehog inhibitor, Cyclopamine (1 μM), for 72 hours reduces the protein expression of Bcl-2. FIG. 12C shows Immunoblots that show that combined exposure for 72 hours to PI3K/AKT inhibitor, Ly294002 (50 μM), and Bcl-2 inhibitor, ABT-737 (10 μM), triggers PARP and Caspase-3 cleavage in DU145- and 22RV1-pCK19-GFP-negative cells. FIG. 12D shows Combined pharmacological inhibition using the same drugs and concentrations as in FIG. 12C decreases colony formation of GFP-negative cells. FIG. 12E shows Flow cytometry analysis after 48-hour administration of Cyclopamine (1 μM) and/or DBZ (1 μM) of DU145-pCK19-GFP and 22Rv1-pCK19-GFP cells stably transfected with empty vector (EV), MYR-AKT, and BCL2. Immunoblots illustrate the overexpression levels of pAKT (Ser-473) and Bcl-2. FIG. 12F shows Colony formation assay and quantification of colonies derived from DU145-pCK19-GFP and 22Rv1-pCK19-GFP CK19/GFP-negative sorted cells stably transfected with empty vector (EV), MYR-AKT, and BCL2 exposed for 72 hours to Cyclopamine at 1 μM (Cyc), GDC-0449 at 1 μM (GDC), DBZ at 1 μM, Compound-E at 1 μM (CE), individually or in combination (Cyc+DBZ or GDC+CE). FIG. 12G shows Colony formation assays and quantification of GFP-positive sorted DU145-pCK19-GFP and 22Rv1-pCK19-GFP stably transfected with EV, MYR-AKT, and BCL2 and treated 72 hours with or without Mitoxantrone (125 nM and 500 nM), Cisplatin (5 μM and 2.5 μM, and Vinorelbine (500 nM and 750 nM), respectively. Data is represented as means±SD of three independent experiments. * corresponds to p<0.05.

FIG. 13A-D: Notch and Hedgehog promote survival and multi-drug resistance in a P-gp/ABCB1 drug efflux independent mechanism. FIG. 13A shows Immunoblots that show that exposure to the Notch inhibitor, CE (1 μM), for 72 hours specifically decreases p-AKT (Ser-473) activity and that exposure to Hedgehog inhibitor, GDC-0449 (1 μM), for 72 hours reduces the protein expression of Bcl-2. FIG. 13B shows Immunoblots that illustrate the expression of the drug efflux pump P-gp/ABCB1 in DU145-pCK19-GFP and 22Rv1-pCK19-GFP sorted cells. Protein lysates of HEK-293 cells transfected with ABCB1 were used as positive controls. FIG. 13C shows Flow cytometry plots that illustrate the detection of a side population (SP) in 22Rv1 cells, but not in DU145 cells. In 22Rv1 cells, the Hoechst 33342 dye efflux is blocked by the P-gp/ABCB1 inhibitor, Verapamil hydrochloride, thus identifying a SP in this cell line. FIG. 13D shows Histograms that illustrate the mRNA expression levels of ABCB1 and Notch (NOTCH2, HES1 and HEY1) and Hedgehog (SMO, GLI1 and GLI2) pathway target genes when treating 22Rv1 CK19/GFP-negative sorted cells with Notch inhibitors (DBZ at 1 μM and CE at 1 μM) and Hedgehog inhibitors (Cyclopamine at 1 μM and GDC-0449 at 1 μM) alone or in combination for 72 hours. Results are displayed relative to vehicle (DMSO) exposed controls. Data is represented as means±SD of three independent experiments. * corresponds to p<0.05.

FIG. 14A-F: The tumor initiating capacity of Docetaxel resistant prostate cancer cells. FIG. 14A shows Tumor incidence, tumor initiating cell (T-IC) frequency, and latencies after 36 weeks of injection of limiting dilutions of parental (DU145 and 22Rv1) and Docetaxel resistant (DU145-DR and 22Rv1-DR) cells. FIG. 14B shows Tumor incidence, T-IC frequency, and latencies after 38 weeks of injection of limiting dilutions of DU145 and 22RV1 HLA class I sorted cells. FIG. 14C shows Picture of a mouse bearing tumors in the upper flanks after injection of DU145 HLA class I-negative cells in the upper flanks and HLA class I-positive cells in the lower flanks H&E and immunofluorescence analysis of CKs (CK18+19), HLA class I, and transcription factors in representative tumor xenografts generated from DU145 and 22RV1 HLA class I-negative cells. White arrows point to CK-negative cells with positive nuclear staining of transcription factors and lack of HLA class I and AR. FIG. 14D shows a Table that summarizes prostate cancer patients' clinico-pathological characteristics, tumor incidence, T-IC frequency, and latencies after 61 weeks of injection of limiting dilutions of HLA class I sorted cells from fresh human prostate cancer samples. FIG. 14E shows an H&E and immunofluorescence analysis of human tumors and primary and secondary xenografts generated from HLA class I-negative cells. Patient #9 is represented in this panel. White arrows point to CK-negative cells with nuclear expression of transcription factors and lack of HLA class I and AR. FIG. 14F shows Tumor incidence and latencies after 24 weeks of injection of 100 HLA class I-negative sorted cells from prostate cancer xenografts treated with DMSO, Dexamethasone 15 mg/kg/ip daily, Cyclopamine 50 μg/kg/sc daily plus Dexamethasone, DBZ 10 μM/kg/ip daily for 15 days every 4 weeks plus Dexamethasone or with the triple combination. Data is represented as means±SD. * corresponds to p<0.05. The bar corresponds to 100 μm.

FIGS. 15A-G: HLA class I-negativity enriches for prostate CK-negative cells, which display a higher tumor initiating capacity in NOD/SCID IL2rg^{-/-} mice and recapitulate the original tumor identity in tumor xenografts, related to FIG. 14. FIG. 15A shows Immunofluorescence analysis and quantification of CKs (CK18+19) and HLA class I in parental DU145 and 22RV1 cells. White arrows point to cells with a CK-negative/HLA class I-negative phenotype. FIG. 15B shows a Representative HLA class I sorting diagram of a primary prostate cancer sample shows that the HLA class I-negative cell compartment represents a minority of the total tumor population. A plot from the same case illustrates that the HLA class I-negative compartment significantly enriches for CK-negative cells, whereas the HLA class I-positive compartment enriches for CK-positive cells. The histogram shows the quantification of CK-negative/HLA class I-negative and CK-positive/HLA class I-positive cells performed in 9 different patient samples. FIG. 15C shows Histograms that display the quantification of immunofluorescence based co-expression analysis of CKs (CK18+19), HLA class I, transcription factors (cleaved Notch-2, Gli1, and Gli2), and androgen receptor (AR) displayed in FIG. 14C. FIG. 15D shows Flow cytometry plots of DU145 cells incubated with an anti-HLA class I antibody alone (control) and with an anti-HLA class I antibody plus complement. Plots illustrate that the addition of complement induces a robust lysis of HLA class I-positive cells (propidium iodide-positive), while HLA class I-negative cells remain viable (propidium iodide-negative). FIG. 15E shows Tumor incidence and latencies after 38 weeks of injection of 100 HLA class I non-depleted and depleted cells. FIG. 15F shows Tumor incidence, T-IC frequency, and latencies after 46 weeks of injection of limiting dilutions of HLA class I sorted cells from primary prostate cancer xenografts injected into NOD/SCID IL2rg^{-/-} mice. Four mice for each sorted cell population and cell dilution were injected twice in the upper flanks (HLA class I-negative) and lower flanks (HLA class I-positive) of mice. (F) Histograms display the quantification of immunofluorescence based co-expression analysis of CKs (CK18+19), HLA class I, transcription factors (cleaved Notch-2, Gli1, and Gli2), and androgen receptor (AR) displayed in FIG. 14E. Data is represented as means±s.d.

FIG. 16: Table of the clinico-pathological characteristics and percentages of HLA class I-negative cells from the 30 injected histologically confirmed fresh human primary prostate tumors. The table, related to FIG. 14, summarizes the clinico-pathological characteristics of the 30 primary prostate cancer patient samples from which HLA sorted cells were used for limiting dilution assays in NOD/SCID mice. Established prognostic factors, such as pre-surgical PSA, tumor differentiation (Gleason score), and extension of the disease (Stage), as well as the mean percentage of HLA class I-negative cells, are represented.

### DETAILED DESCRIPTION

The present technology is based upon the discovery of a phenotype that is resistant to chemotherapy agents, e.g., a Docetaxel resistance phenotype that is characterized by absence of epithelial differentiation markers and HLA class I antigens, as well as activation of developmental pathways.

Using a Docetaxel resistance model in HRPC cells, a population of prostate cancer cells that exhibits resistance to Docetaxel were identified. The Docetaxel resistant cells had an undifferentiated phenotype, dependence on combined Notch and Hedgehog signaling, and high tumor initiating capacity. In vitro studies showed that this Docetaxel resistance phenotype corresponds to a small, intrinsically multi-drug resistant subpopulation present in unselected HRPC cells. Interestingly, this drug resistant subpopulation was significantly higher in metastatic patients treated with Docetaxel than in untreated patients. Further, the abundance of cells exhibiting the Docetaxel resistance phenotype was higher in metastatic than primary samples, suggesting an association with tumor aggressiveness. Additionally, in primary untreated samples, the percentage of this resistant population strongly predicted time to biochemical relapse, which is predictive of survival time of prostate cancer.

The subpopulation of cells, which exhibits the characterized Docetaxel resistance phenotype in cell lines and prostate cancer tissues, possessed properties of tumor initiating cells. Tumor initiating cells are thought to possess the capacity to self-renew and generate the diversity of cells that comprise a tumor. As these tumor initiating cells may contribute to disease progression by participating in chemotherapy resistance, the present methods are advantageous in that they would diminish this population of tumor initiating cells.

The Docetaxel resistant cells exhibited upregulation of Notch and Hedgehog signaling. Genetic knockdown studies demonstrated that both Notch and Hedgehog pathways in combination are critically important for the maintenance of Docetaxel resistant cells. Using clinically viable pharmacological inhibitors of these pathways, it was determined that a combination strategy utilizing a combination of inhibitors successfully depleted Docetaxel resistant cells. Notably, the combination of Notch and Hedgehog inhibitors with Docetaxel in vivo abrogated tumor regrowth after Docetaxel administration. In addition, inhibition of the Notch and Hedgehog signaling pathways significantly reduced tumor incidence in mouse xenografts models of prostate cancer.

Mechanistically, it was observed that Notch signaling regulated the activation of the PI3K/AKT pro-survival pathway, while Hedgehog signaling upregulated anti-apoptotic pathway molecules. Specifically, Notch signaling led to increased phosphorylation of AKT (Ser473), leading to increased levels of p-AKT, and Hedgehog signaling upregulated Bcl-2 expression levels. Combined inhibition of PI3K/AKT and Bcl-2 mimicked combined Notch and Hedgehog blockage by depleting Docetaxel resistant cells. Overexpression of PI3K/AKT and Bcl-2 pathway molecules rescued Docetaxel resistant cells from Notch and Hedgehog inhibition and conferred multidrug resistance to normally sensitive cells. The present technology provides a set of newly-identified targets for combating resistance to chemotherapy agents in HRPC, a widespread and fatal disease. Specifically, provided herein are methods to effectively prevent or reduce such resistance in cancers such as prostate cancer.

Accordingly the present technology features methods of preventing or reducing resistance to one or more chemotherapy agents (including but not limited to Docetaxel) in cancer (including but not limited to prostate cancer) by, e.g., administering to a subject a Notch signaling pathway inhibitor and/or a Hedgehog signaling pathway inhibitor. Optionally the subject is further treated with an additional amount of one or more chemotherapy agents (including but not limited to Docetaxel). In certain embodiments, the additional chemotherapy agent (which may or may not be the same chemotherapy agent that the cells are shown to be resistant to) is administered to the subject prior to, after or concurrently with the Notch signaling pathway inhibitor and/or a Hedgehog signaling pathway inhibitor. The subject is for example a human having cancer (including but not limited to prostate cancer) who has not been treated for cancer. Alternatively, the subject is a human having cancer and has been treated for cancer. Treatment may include Docetaxel. In certain embodiments, the subject has hormone refractory prostate cancer (HRPC). In some aspects the subject is known to be resistant to any chemotherapy agent, including but not limited to Docetaxel.

Also included in various embodiments of the present technology are methods of identifying a population of tumor cells that are resistant to one or more chemotherapy agents (including but not limited to Docetaxel resistant tumor cells) by detecting activation of the Notch or Hedgehog signaling pathways, downregulation of HLA class I antigens, or downregulation of cytokeratin 18 or cytokeratin 19. In certain embodiments, activation of Notch or Hedgehog signaling pathways and/or downregulation of HLA class I antigens cytokeratin 18 or cytokeratin 19 or any combination thereof indicates a tumor cell that is resistant to one or more chemotherapy agents (including but not limited to Docetaxel resistant tumor cells).

In other embodiments, the present technology provides methods of predicting survivability of cancer by detecting activation of the Notch and Hedgehog signaling pathways, downregulation of HLA class I antigens, cytokeratin 18 or cytokeratin 19 or any combination thereof. In certain embodiments, the technology provides methods of predicting overall survival or recurrence free survival or response to therapy of subjects having cancer by detecting activation of the Notch and Hedgehog signaling pathways, downregulation of HLA class I antigens, or downregulation of cytokeratin 18 or 19.

In certain embodiments, activation of the Notch and Hedgehog signaling pathways, downregulation of HLA class I antigens, or downregulation of cytokeratin 18 or cytokeratin 19 or any combination thereof indicates decreased survival time, tumor aggressiveness and/or poor patient prognosis.

In certain embodiments, the present technology may be used to make continuous or categorical measurements of the response to chemotherapy or cancer survival, thus diagnosing and defining the risk spectrum of a category of subjects defined as at risk for not responding to chemotherapy, such as, but not limited to, Docetaxel. In the categorical scenario, in certain embodiments the methods of the present technology are used to discriminate between treatment responsive and treatment non-responsive subject cohorts. In other embodiments, the present technology may be used so as to discriminate those who have an improved survival potential.

Identifying the subject who will be responsive to therapy permits the selection and initiation of various therapeutic interventions or treatment regimens in order increase the individual's survival potential. Levels of molecules involved in the Notch and Hedgehog signaling pathways, HLA class I antigens, cytokeratin 18 or cytokeratin 19 allow for the course of treatment of a metastatic disease or metastatic event to be monitored. In such methods, a biological sample can be provided from a subject undergoing treatment regimens, e.g., drug treatments, for cancer such as prostate cancer. If desired, biological samples are obtained from the subject at various time points before, during, or after treatment.

Levels of molecules involved in the Notch and Hedgehog signaling pathways, HLA class I antigens, cytokeratin 18 or cytokeratin 19 can be determined and compared to a reference value, e.g., a control subject or population whose therapeutic responsiveness is known or an index value or baseline value. In various embodiments, the reference sample or index value or baseline value may be taken or derived from one or more subjects who have been exposed to treatment, or may be taken or derived from one or more subjects who are at low risk of surviving the cancer, or may be taken or derived from subjects who have shown improvements in as a result of exposure to treatment. Alternatively, the reference sample or index value or baseline value may be taken or derived from one or more subjects who have not been exposed to the treatment. For example, samples may be collected from subjects who have received initial treatment for cancer or and subsequent treatment for cancer or a metastatic event to monitor the progress of the treatment.

Various techniques may be used in survival and time to event hazard analysis, including Cox, Weibull, Kaplan-Meier and Greenwood models well known to those of skill in the art.

Detection of activation of Notch or Hedgehog signaling may include detection of cleaved Notch2, increased expression of Hes1, increased expression of Hey1, increased expression of Gli1, increased expression of Gli2, increased expression of Smo, reduced expression of Patched, phosphorylation of AKT (Ser473), and increased expression of Bcl-2. Increased or reduced expression of the above-mentioned molecules is relative to the expression levels in non-Docetaxel resistant cells. HLA class I antigens may include A, B, C, E, F and G.

A Notch inhibitor is a compound that decreases expression or activity of molecules in the Notch signaling pathway. Notch inhibitors include, for example, DBZ [(2S)-2-[2-(3,5-difluorophenyl)-acetylamino]-N-(5-methyl-6-oxo-6,7-dihydro-5H-dibenzo[b,-d]azepin-7-yl)-propionamide], Gamma Secretase Inhibitor (GSI-18) (WO2007100895A2), the gamma secretase inhibitor L-685,458 (CAS 292632-98-5), gamma-secretase inhibitor MW 167 (Calbiochem gamma-secretase inhibitor II, Cat. No. 565755), L-685,458 (Shearman, M. S. et al., Biochem. 39: 698-8704 (2000)), Compound E (CAS 209986-17-4), or PI3K/AKT pathway inhibitors. Alternatively, a Notch inhibitor is a nucleic acid that inhibits the expression or activity of one or more molecules in the Notch signaling pathway, for example, Notch2. In one embodiment, the nucleic acid is an antisense nucleic acid, short hairpin RNA, or small interfering RNA. In another embodiment, a Notch inhibitor is a peptide or polypeptide. In an embodiment, the Notch inhibitor is an antibody or antibody fragment. Other suitable inhibitors are described in EP1718767B1, U.S. Pat. No. 7,544,476, WO2003041735A2, WO2007029030A2, and WO2012068477A1, the contents of which are incorporated by reference in their entireties.

A Hedgehog inhibitor is a compound that decreases expression or activity of molecules in the Hedgehog signaling pathway. Hedgehog inhibitors include, for example, Cyclopamine, GDC-0449 (Vismodegib), or Bcl-2 family member inhibitors. Other suitable inhibitors are described in WO2005042700A2, the content of which is incorporated by reference in its entirety.

Alternatively, a Hedgehog inhibitor is a nucleic acid that inhibits the expression or activity of one or more molecules in the Hedgehog signaling pathway, for example, Gli1 or Gli2. In one embodiment, the nucleic acid is an antisense nucleic acid, short hairpin RNA, or small interfering RNA. In another embodiment, a Hedgehog inhibitor is a peptide or polypeptide. In an embodiment, the Hedgehog inhibitor is an antibody or antibody fragment.

Inhibitors for PI3K/AKT and Bcl-2 include but are not limited LY294002 [2-(4-Morpholinyl)-8-phenyl-1(4H)-benzopyran-4-one hydrochloride] and ABT-737 (CAS 852808-04-9), respectively. Other suitable inhibitors are described in US 2012/0189539A1 the content of which is incorporated by reference in its entirety.

In some aspects of the present technology, cells are contacted with, or a subject is treated with, a combination of inhibitors. An exemplary combination of inhibitors is one or more Notch inhibitor with one or more Hedgehog inhibitor. In one embodiment, the combination of inhibitors is one Notch inhibitor and one Hedgehog inhibitor. For example, an effective combination is an shRNA against Notch2 and an shRNA against Gli1 or Gli2. Another effective combination is Cyclopamine plus DBZ. Another effective combination is GDC-0449 plus Compound E. Yet another effective combination is LY294002 plus ABT-737. In another embodiment, the combination of inhibitors is one Notch inhibitor and two Hedgehog inhibitors. For example, an effective combination is an shRNA against Notch2 plus an shRNA against Gli1 plus an shRNA against Gli2. Another effective combination is Compound E plus Cyclopamine and GDC-0449. In another embodiment, the combination of inhibitors is two Notch inhibitors and one Hedgehog inhibitor. An effective combination is DBZ and Compound E plus Cyclopamine. Optionally, the combinations of inhibitors mentioned herein are administered with one or more chemotherapy agents, e.g., Docetaxel, before, after, or concurrently. Optionally, combinations of inhibitors are administered with other drugs, such as Dexamethasone, before, after, or concurrently. An example of such an effective combination is Docetaxel with Cyclopamine plus DBZ plus Dexamethasone.

Downregulation, upregulation, and changes in expression and levels of molecules described herein may refer to mRNA or protein. Expression levels can be assessed by any methods known in the art or by using methods described herein.

As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

The compounds, e.g., Notch2 or Hedgehog inhibitors of the present technology, and derivatives, fragments, analogs and homologs thereof, can be incorporated into pharmaceutical compositions suitable for administration. Such compositions typically comprise the compound, and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers include those described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is known in the art. Except insofar as any conventional media or agent is incompatible with the active compound, use thereof in the compositions is contemplated. Supplementary active compounds can also be incorporated into the compositions.

In various embodiments, a pharmaceutical composition of the present technology is formulated to be compatible with its intended route of administration. Examples of routes of administration include parenteral, e.g., intravenous, intradermal, subcutaneous, oral (e.g., inhalation), transdermal (topical), transmucosal, and rectal administration. In an exemplary embodiment, solutions or suspensions used for parenteral, intradermal, or subcutaneous application include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. In one aspect, the parenteral preparation is enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In certain embodiments, the composition should be sterile and should be fluid to the extent that easy syringeability exists. It should be stable under the conditions of manufacture and storage and should be preserved against the contaminating action of microorganisms such as bacteria and fungi. In one aspect, the carrier is a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In certain embodiments, isotonic agents can be included, for example, sugars, polyalcohols such as manitol, sorbitol, sodium chloride in the compositions. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation include vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In certain embodiments, for administration by inhalation, the compounds can be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants include, for example, for transmucosal administration, detergents, bile salts, and fusidic acid derivatives. Transmucosal administration can be accomplished through the use of nasal sprays or suppositories. For transdermal administration, the active compounds can be formulated into ointments, salves, gels, or creams.

In certain embodiments, the compounds can also be prepared in the form of suppositories (e.g., with conventional suppository bases such as cocoa butter and other glycerides) or retention enemas for rectal delivery.

In certain embodiments, the active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, incorporated fully herein by reference.

In certain embodiments, it is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the present technology are dictated by and directly dependent on the unique characteristics of the active compound and the particular therapeutic effect to be achieved.

In certain embodiments, the pharmaceutical compositions can be included in a container, pack, or dispenser together with instructions for administration.

### EXAMPLES

The following examples, including the experiments conducted and results achieved, are provided for illustrative purposes only and are not to be construed as limiting upon the present technology.

### Example 1

### General Methods

Generation of Acquired Docetaxel Resistant Prostate Cancer Cell Models.

Human HRPC cell lines, DU-145 and 22RV1, were obtained from American Type Culture Collection (ATCC) and maintained in RPMI 1640 medium (Gibco) supplemented with 10% FBS without antibiotics. Docetaxel resistant clones, DU-145-DR and 22RV1-DR, were selected by culturing cells with Docetaxel in a dose-escalation manner using 72 hr exposures. Initial culture was at 5 nM Docetaxel for DU145 and 25 nM for 22RV1. After sensitive clones were no longer present and surviving DU-145 and 22RV1 cells repopulated the flask, the concentration of Docetaxel was increased to 10 nM, 25 nM, 50 nM, 100 nM and 250 nM. 22RV1-DR cells were further exposed to 500 nM Docetaxel. The process of acquired drug resistance took 9 months for DU-145-DR and 6.5 months for 22RV1-DR. In parallel, parental DU-145 and 22RV1 cells were exposed to DMSO (vehicle solution) in the same dose-escalation manner.

Human Prostate Cancer Tissue Samples.

Formalin-fixed paraffin-embedded human primary (n=31) and metastatic (n=36) prostate cancer tissue samples were provided by the tumor bank of Columbia University Cancer Center. Fresh primary prostate tumor tissue samples (n=30) were obtained from patients who had undergone surgical procedures at Columbia University Medical Center. All samples were collected under informed consent and under the supervision of the Columbia University Medical Center Institutional Review Board. Tissue sections with cancer were selected by reviewing Hematoxylin & Eosin (H&E) stained slides.

Mouse Procedures.

Animal use and care was in strict compliance with institutional guidelines established by the University of Columbia, Institutional

Animal Care and Use Committee. Xenograft experiments were performed with 5-6 weeks old NOD.Cg-Prkdc^{scid} IL2rg^{tm1Wj1}(NSG) and NOD.CB17-Prkdc^{scid} (NOD/SCID) mice obtained from Jackson Laboratories.

Accession Numbers.

Microarray data were deposited at GEO with the accession number GSE36135.

Targeted Pathway Inhibitors and Drugs.

Docetaxel, Mitoxantrone, Cisplatin, Vinorelbine, Dexamethasone, Cyclopamine, and Compound E (CAS 209986-17-4) were obtained from Sigma-Aldrich. DBZ [(2S)-2-[2-(3,5-difluorophenyl)-acetylamino]-N-(5-methyl-6-oxo-6,7-dihydro-5H-dibenzo[b,-d]azepin-7-yl)-propionamide] was obtained from Syncom. The Hedgehog inhibitor GDC-0449 (Vismodegib), the selective PI3K/AKT pathway inhibitor LY294002, and Bcl-2 family member inhibitor ABT-737 were obtained from Selleck.

Cell Viability and Colony Formation Assays.

Cell viability was analyzed using the Cell titer 96 Aquos Non-Reactive Cell Proliferation Assay (MTs) kit (Promega). Cells were seeded at a density of 10,000 in 96-well culture dishes, and 24 hours later, medium was removed and replaced with new medium alone (control) or medium containing drugs. After 72 hours, color absorbance was measured on a microplate spectrophotometer (Molecular Dynamics) at 450 nm (test wavelength) and 620 nm (reference wavelength). The percentage of surviving cells was estimated by dividing the A450 nm-A620 nm of treated cells by the A450 nm-A620 nm of control cells. Clonogenic survival assays in response to drug treatment were performed by plating approximately 1,000 cells in 35 mm culture dishes. After 24 hours, cells were left untreated (control) or treated with drugs. The next day, medium was changed, and the cells continued growing in fresh medium without any drug or under exposure to drugs. For continuous exposure experiments, medium plus drugs was replaced every 3 days until clones of drug-resistant cells appeared. Cells were then fixed with 4% paraformaldehyde in PBS, stained with crystal violet solution and formed colonies were visually counted.

Analysis of Apoptosis by Flow Cytometry.

Cells (100,000) were left untreated (control) or treated with drugs for 72 hours. Adherent and detached cells were pooled, washed, and labeled with Annexin-V-FITC and propidium iodide using the Annexin-V-FLUOS Staining Kit (Roche) according to manufacturer's instructions. Samples were acquired with a FACscan Flow Cytometer (BD Biosciences) and analyzed with CellQuest Pro software (BD Biosciences) to determine the percentage of cells displaying Annexin V staining.

cDNA Microarray Analysis.

22RV1, 22RV1-DR, DU-145, and DU-145-DR gene expression profiles were analyzed. Total RNA from each sample was isolated by Trizol (Invitrogen) and purified by RNeasy Mini kit and RNase-free DNase set (Qiagen) according to the manufacturer's protocols. RNA quality of all samples was tested by RNA electrophoresis and RNA LabChip analysis (Agilent) to ensure RNA integrity. Samples were prepared for analysis with Affymetrix Human U133 arrays according to the manufacturer's instructions. Gene expression levels of samples were normalized and analyzed with Microarray Suite, MicroDB, and Data Mining tool software (Affymetrix). The absolute call (present, marginal, or absent) and average difference of 22,215 expressions in a sample, and the absolute call difference, fold change, and average difference of gene expression between two or three samples were normalized and identified using this software package. Statistical analysis of the mean expression average difference of genes, which show ≧1.8-fold change based on a logarithmic normalization, was done using a t-test between Docetaxel sensitive and resistant samples. Genes that were not annotated or not easily classified were excluded from the functional clustering analysis.

Gene Ontology Analysis.

Genes differentially expressed in the Docetaxel resistant cells compared to the parental sensitive cells generated a list of commonly deregulated transcripts. This list was assessed by the DAVID Bioinformatics Resources, a web-based statistical hypergeometric test applied for enrichment analysis of gene ontology (GO) categories, which include biological process, molecular function, and cellular component (http://david.adcc.ncifcrf.gov/). GO categories enriched on the highest hierarchical level (≧level 5) at statistical significance (p<0.01) were taken into consideration.

Immunoblot Analyses.

Whole cell extracts were prepared in sample buffer and analyzed by immunoblotting. Primary antibodies against poly (ADP-ribose) polymerase (PARP) (BD Pharmingen), cleaved caspase-3 (Cell Signaling), cytokeratin 19 (CK19) (Abcam), cytokeratin 18 (CK18) (Abcam), androgen receptor (AR) (Sigma-Aldrich), prostate specific membrane antigen (PSMA) (Abcam), prostate specific antigen (PSA) (Epitomics), cytokeratin 5 (CK5) (Covance), cytokeratin 14 (CK14) (Biogenex), p63 (Santa Cruz), CD44 (Neomarkers), activated Notch2 (Abcam), Hes1 (Abcam), Patched (Abcam), Gli1 (Santa Cruz), Gli2 (Abcam), pan-HLA class I (Abcam), and AKT (Cell Signaling), phospho-AKT Ser 473 (Cell Signaling), Bcl-2 (Cell Signaling), ABCB1/P-Glycoprotein (Calbiochem), Green fluorescent protein (Santa Cruz), and β-Actin (Sigma-Aldrich) were used in immunoblot assays using standard procedures. Protein expression was quantified by comparing band expression using Quantity One software (Bio-Rad).

Generation of the Cytokeratin 19-Green Fluorescent Protein (GFP) Reporter Plasmid.

CK19 gene promoter region was amplified from genomic DNA of DU145 cells by PCR with specific primer sets (Fw 5′-AACGCATGCTTTGGGGGGATG-3′ and Rv 5′-TCCCCCTTTACTCGGCCCCCAC-3′) as described previously (Tripathi et al., 2005). Briefly, a region of 1768 bp corresponding to human CK 19 promoter was amplified. The promoter region includes 1142 bp of the 5′ UTR region, 480 bp belonging to Exon 1 and 146 bp belonging to Intron 1. The PCR products were digested with Ase 1 and Hind 111 and cloned into pEGFPN1 vector (Clontech) previously digested with the same enzymes. As a result, the CMV promoter was removed from the original vector and the GFP expression was under the control of the CK19 promoter. The final construct was confirmed by digestion and sequencing analysis. DU145 and 22Rv1 cells were transfected with the pCK19-GFP construct using Lipofectamine Plus 2000 (Invitrogen). After 24 hours, medium was replaced with fresh medium and stably expressing cells selected in the presence of G418 (Invitrogen). Positive clones were confirmed by direct microscopy and immunofluorescence and also by PCR amplification of the GFP coding region using specific primers (Fw 5′-TTCCTGCGTTATCCCCTGATTC-3′ and Rv 5′-GCTCCTCCGGCCCTTGCTCACCAT-3′).

RT-PCR and Quantitative RT-PCR.

Total RNA was isolated using the RNeasy Mini kit according to the manufacturer's instructions (Qiagen). Complementary DNA was synthesized from equivalent concentrations of total RNA using the SuperScript III First-Strand Synthesis SuperMix Kit (Invitrogen) according to the manufacturer's instructions. Coding sequences for genes of interest and β-Actin as an internal control were amplified from 500 ng of complementary DNA using the QuantiTect SYBR Green PCR Kit (Qiagen). Custom primer sequences used for amplification experiments are shown in Table 1.

TABLE 1

| | Forward Sequence | Reverse Sequence |
|---|---|---|
| Gene | (5′ to 3′) | (5′ to 3′) |
| CD34 | ACAAACATCACAGAAA | TGACAGGCTAGGCTTC |
| | CGACAGT | AAGGT |
| CDH2 | ATCGCATTATGCAAGA | ATGCACATCCTTCGAT |
| | CTGGATT | AAGACTG |
| CDH5 | TTGGAACCAGATGCAC | TCTTGCGACTCACGCT |
| | ATTGAT | TGAC |
| ABCB1 | TGTTCAAACTTCTGCT | CCCATCATTGCAATAG |
| | CCTGA | CAGG |
| CK18 | GAGACGTACAGTCCAG | CCACCTCCCTCAGGCT |
| | TCCTTGG | GTT |
| CK19 | CTGCGGGACAAGATTC | CCAGACGGGCATTGTC |
| | TTGGT | GAT |
| GLI1 | CCCAACTCCACAGGCA | ACACGAACTCCTTCCG |
| | TAC | CTCC |
| GLI2 | ACACCAACCAGAACAA | AGTCTTCCCAGTGGCA |
| | GCAG | GTTG |
| HES1 | CTGGAAATGACAGTGA | ATTGATCTGGGTCATG |
| | AGCACCT | CAGTTG |
| HEY1 | GCTGGTACCCAGTGCT | TGCAGGATCTCGGCTT |
| | TTTGAG | TTTCT |
| HLA-A | AAAAGGAGGGAGTTAC | GCTGTGAGGGACACAT |
| | ACTCAGG | CAGAG |
| HLA-B | CAGTTCGTGAGGTTCG | CAGCCGTACATGCTCT |
| | ACAG | GGA |
| NOTCH2 | GGCATTAATCGCTACA | GGAGGCACACTCATCA |
| | GTTGTGTCT | ATGTCA |
| PTCH1 | TTGCTTGGGAGTCATT | CCCACAATCAACTCCT |
| | AACTGG | CCTGCC |
| SMO | ATGGATGGTGCCCGCC | ATGGTCTCGTTGATCT |
| | GAGAG | TGCTGG |
| β-ACTIN | AAACTGGAACGGTGAA | GTGGCTTTTAGGATGG |
| | GGTG | CAAG |

Amplification was carried out using a Stratagene mx3005p (Agilent Technologies). Cycle threshold (Ct) values were determined and normalized to the housekeeping gene (β-actin) for each experiment. Fold changes for experimental groups relative to respective controls were calculated using MX Pro software (Agilent Technologies). Quantitative RT-PCR was performed in DU145-pCK19-GFP and 22Rv1-pCK19-GFP cells as well as in cells obtained from mice bearing DU145 and 22Rv1 tumor xenografts treated for 21 days with Docetaxel, and Hedgehog and Notch inhibitors, alone or in combination. Quantitative RT-PCR was performed in tumor xenografts removed 4 hours after the last drug dose was administered.

Short Hairpin RNA Knockdowns.

The expression of human Notch and Hedgehog critical pathway genes were knocked down using the inducible shRNA-mirs from Open Biosystems listed in Table 2. Sequences for these shRNAs are available at http://www.openbiosystems.com.

TABLE 2

| Target | shRNA |
|---|---|
| NOTCH2 | V2THS_135984 |
| | V2THS_135984 |
| GLI1 | V2LHS_42494 |
| | V3LHS_387772 |
| GLI2 | V3LHS_321206 |
| | V3LHS_321208 |

The inducible vector containing a scrambled sequence that doesn't target any human sequence was used as a control (Empty Vector). Every shRNA was transfected individually in a packaging cell line, and the obtained lentivirus was used to infect the different prostate cancer cell lines at a high MOI (>95% infection efficiency). Selection with Puromycin was done two days upon infection, and cells expressing high amounts of the shRNA were sorted after 24 h of induction with Doxycycline (50 ng/ml).

Immunohistochemistry and Immunofluorescence Analyses.

Immunofluorescence analyses were conducted on prostate cancer cell lines and formalin fixed paraffin-embedded tissue sections from normal human prostate, human cancers, and tumor xenografts. Primary antibodies included a combination of cytokeratin 19 and 18 (Abcam), green fluorescence protein (Abcam), cytokeratin 5 (Covance), cytokeratin 14 (Biogenex), p63 (Santa Cruz), pan-HLA class I (Abcam), Ki67 (Abcam) and the following transcription factors: activated Notch2 (Abcam), Gli1 (Santa Cruz), Gli2 (Abcam) and androgen receptor (DAKO). Secondary antibodies labeled with Alexa Fluor® 594 (Invitrogen) and Alexa Fluor® 488 (Invitrogen) were used. Prostate cancer cells (10,000) were plated in 35 mm culture dishes and 24 hours later stained by standard immunofluorescence procedures. Tissue sections (5 μm) were deparaffinized and submitted to standard peroxidase based immunohistochemistry and immunofluorescence procedures. Quantification of the expression of cytokeratins, HLA class I antigens, transcription factors, and androgen receptor was performed by evaluating tumor cells. Percentage of positive and negative cells was determined by counting the number of tumor cells in 10 contiguous high power fields in three different areas of the tumor, and referred to the total number of counted cancer cells.

In Vitro Effects of Notch and Hedgehog Pathway Inhibitors.

The in vitro effects of Notch and Hedgehog inhibitors on DU145-pCK19-GFP and 22Rv1-pCK19-GFP cell lines were analyzed by flow cytometry and colony formation assays (described above). Cells were exposed to vehicle solution (Control), Cyclopamine (1 μM), GDC-0449 (1 μM), Compound E (1 μM), DBZ (1 μM), and a dual combination (e.g. Cyclopamine plus Compound E), or a triple of Docetaxel and one of each developmental pathway inhibitors (e.g. Docetaxel plus Cyclopamine plus Compound E).

In Vitro Effects of PI3K/AKT and Bcl-2 Pathway Inhibitors.

The in vitro effects of the selective PI3K/AKT inhibitor, LY294002, and Bcl-2 family member inhibitor, ABT-737, on DU145-pCK19-GFP and 22Rv1-pCK19-GFP cell lines were analyzed by colony formation and immunoblotting assays (described above). Cells were exposed to vehicle solution (Control), LY294002 (50 μM), and ABT-737 (10 μM) for 72 hours, alone or in combination.

Generation of the Bcl-2 and MYR-AKT Overexpression Plasmid.

Bcl-2 cDNA was amplified by PCR from plasmid DNA with specific primers (Fw 5′-AAAAAGAATTCCGCCACCATGGCGCACGCTGGGAGAACA-3′ and Rv 5′-AAAAGCGGCCGCTCACTTGTGGCCCAGATAGGC-3′). The 720 bp amplicon was digested with EcoRI and NotI restriction enzymes for 2 h at 37° C. and cloned into the pLPCX (Clontech) vector, which was previously digested and dephosphorylated. The final construct was confirmed by digestion and sequencing analysis. Retroviral particles driving the expression of myristoylated AKT (MYR-AKT) were a gift from Dr. Adolfo Ferrando (Columbia University, New York). The vector containing a scrambled sequence that does not target any human sequence was used as a control (Empty Vector). Both DU145-pCK19-GFP and 22Rv1-pCK19-GFP cells were infected with virus containing BCL-2, MYR-AKT, and empty vector, and selected with puromycin (Sigma) to generate stable cell lines.

Analysis of Side Population Cell Fractions.

DU145 and 22RV1 cells were used in the side-population assay to identify cells that overexpress ABC transporters, which enables them to transport Hoechst 33342 dye. Briefly, cells were suspended in pre-warmed RPMI 1640 medium (Gibco) supplemented with 2% FBS at 1×10⁶ cells/ml and incubated with Hoechst 33342 dye (Invitrogen) to a final concentration of 5 μg/ml for 90 minutes at 37° C. Control cells were incubated with 50 μM verapamil hydrochloride (Sigma) for 15 minutes at 37° C. before Hoechst 33342 dye addition to inhibit the ABCB1 transporters. All cells were immediately placed on ice, washed, and resuspended in ice-cold PBS containing 2% FBS. Dead cells were excluded from the analysis by propidium iodide (Sigma) staining Hoechst 33342 labeled cells were analyzed on a LSRII flow cytometer (BD Biosciences). Side population cells were visualized by use of red (FL8) versus blue (FL7) ultraviolet channels.

Live Cell Imaging.

Time-lapse videomicroscopy was used to assess Docetaxel sensitivity of DU145-pCK19-GFP and 22Rv1-pCK19-GFP cells. Cells growing in 6-well plates at low confluence were placed in the stage inside an incubator chamber at 37° C., 50% humidity, and in an atmosphere of 5% CO₂. Unattended time-lapse movies of randomly chosen GFP+ and GFP− DU145 and 22Rv1 cells were performed with a Nikon Eclipse Ti inverted microscope. NIS Elements AR (Nikon) software was used to collect and process data. Imaging was performed using a 10× objective, and images were captured using 200-ms exposure times for GFP and 20-ms exposure times for bright field every 30 min.

Analysis of Subpopulations of Cells by Flow Cytometry.

Flow cytometry analysis of subpopulations of prostate cancer cells were carried out following standard procedures. Intracellular CK19 and CK18 expression analysis was performed in single-cell suspensions fixed with 70% ethanol, whereas the expression of cell surface HLA class I was determined in fresh cell samples (without fixation). Primary antibodies against CK19 (Abcam), CK18 (Abcam), HLA class I (Abcam), HLA class I conjugated to phycoerythrin (Abcam), and GFP (Abcam) were used. Secondary antibodies, when used, corresponded to Alexa Fluor® 594 (Invitrogen) and Alexa Fluor® 488 (Invitrogen) labeled immunoglobulins. Samples were acquired with a FACscan Flow Cytometer (BD Biosciences) and analyzed with CellQuest Pro software (BD Biosciences). A minimum of 10,000 cells were measured per sample.

Complement Lysis.

DU145 and 22RV1 cells were incubated for 30 min at 37° C. with an IgG2a mouse antibody directed against HLA-class I (Abcam) in HBSS medium (Gibco) supplemented with 10% FCS. After washing twice with PBS, cells were cultured at 37° C. for 45 min in HBSS without FCS containing a 1:5 dilution of rabbit complement (Accurate Chemical & Scientific). Cell purity was checked using standard flow cytometry by comparing the percentage of cells expressing HLA class I before and after complement depletion.

In Vivo Effects of the Combination of Docetaxel with Notch and Hedgehog Pathway Inhibitors.

To assess the combined effects of Docetaxel with Notch and Hedgehog inhibitors in vivo, subcutaneous xenografts of hormone-independent metastatic prostate cancer cell lines, DU145 and 22RV1, were generated by injecting 2×10⁶ cells embedded in matrigel (BD Biosciences) into the upper flanks of immunodeficient NOD/SCID mice. Once tumors reached a volume in the range of 150-200 mm³, mice were randomly assigned to treatment groups containing eight animals. Treatment groups consisted of Dexamethasone alone (15 mg/kg/ip daily); a double drug combination of Cyclopamine (50 μg/kg/sc daily), DBZ (10 μM/kg/ip daily for 15 days every 4 weeks) or Docetaxel (10 mg/kg/ip once a week for 3 weeks every 4 weeks) with Dexamethasone; a triple drug combination (e.g. Dexamethasone, Docetaxel, and Cyclopamine); and a quadruple combination of drugs, which included Dexamethasone, Docetaxel, Cyclopamine, and DBZ. In parallel, the same treatment groups were treated with Etoposide (10 mg/kg/iv once a week for 3 weeks every 4 weeks) instead of Docetaxel in order to control for multidrug toxicity.

Tumor growth was measured weekly using Vernier calipers. Tumor volume was calculated according to the formula: V=(a²×b)/2, where a and b are the minimal and maximal diameter in millimeters, respectively. Fold-change in tumor volume after initiation of treatments was calculated as: volume at each time point/initial volume. When tumors reached approximately a 3-fold increase from their initial tumor volume, mice were sacrificed. Tumors of sacrificed mice were excised and histologically confirmed. Mice body weight was also recorded weekly, and percentage of mice body weight during treatment was calculated as: (weight at each time point/initial weight)×100. For animals that showed signs of toxicity (mucous diarrhea, abdominal stiffness, and weight loss), drug treatment was discontinued until resolution of the toxicity, and in the next treatment cycle, 50% of the initial drug dose was administered.

Assessment of Tumor Initiating Capacity by Limiting Dilution Assays.

To compare the tumor initiating capacity of Docetaxel sensitive parental cells versus Docetaxel resistant cells and HLA class I-positive versus HLA class I-negative cells, different dilutions of sorted cells (e.g., 10, 100, 1000, and 10,000 cells) were subcutaneously injected in 200 μL of medium: Matrigel (1:1) into male mice. For HLA class I cell isolation, cells were blocked with PBS+FBS 5% and stained with an HLA class I antibody directly conjugated to phycoerythrin (Abcam). Three independent experiments were performed, each one including four NOD/SCID IL-2 receptor gamma chain null (IL2rg^{-/-}) mice injected in both upper flanks with Docetaxel resistant and HLA class I-negative cells and both lower flanks with parental and HLA class I-positive cells, respectively, for each cell dilution and cell line.

To assess the tumor initiating capacity of human cancer cells from fresh tumor tissue samples, portions of tumors were obtained from patients who had undergone surgical procedures at Columbia University Medical Center through an Institutional Review Board approved protocol. Thirty histologically confirmed primary prostate cancers were processed. Specimens were mechanically dissociated and filtered to obtain a single-cell suspension and exposed to red cell lysis buffer (Sigma-Aldrich) to remove red blood cells. Cells were stained with directly conjugated fluorescent antibodies to human CD45 (Abcam), human CD31 (eBiosciences), and human HLA class I (Abcam). For xenograft tumors, primary fluorescent conjugated antibodies to mouse CD45 (eBiosciences), mouse CD31 (Biolegend), mouse HLA class I (Fitzgerald), and human HLA class I (Abcam) were used to select live human cancer cells. Cells were suspended in 10 μg/ml DAPI to label dead cells and sorted on a FACSAria Cell Sorting System (BD Biosciences). In a subset of human primary prostate cancer samples (n=9) with macroscopically identifiable tumor nodules, a fraction of HLA sorted cells was fixed with ethanol (70%) to assess intracellular expression of CKs 18 and 19 by conventional flow cytometry. HLA sorted cells (HLA class I-negative and HLA class I-positive) from human fresh tissue samples (primary injections) and derived xenografts (secondary injections) were injected into NOD/SCID and NSG mice. Four mice for each sorted cell population were injected. Four injections were performed in each mouse for sorted cells, two in the upper flanks for HLA class I-negative cells and two in the lower flanks for HLA class I-positive cells. Secondary injections of HLA sorted cells were performed from tumors generated from HLA class I-negative sorted cells. Tumor initiation was measured by tumor incidence (number of tumors/number of injections) and latency (time from injection to first tumor palpability). Tumor formation was evaluated regularly by palpation of injection sites. In cases in which a tumor became palpable at only one injection site, that tumor was surgically removed to allow continued evaluation of other injection sites. Mice were monitored for up to 62.0 weeks. Animals with no sign of tumor burden were also examined on necropsy to confirm that there was no tumor development. Tumors harvested were fixed in formalin, and paraffin sections were cut for H&E staining and immunohistochemistry/immunofluorescence studies when necessary.

To calculate the average frequency of tumor initiating cells in the above experiments, the maximum-likelihood estimation method of limiting dilution assay (O'Brien et al., 2007; Porter and Berry, 1963) was used. Internal consistency was validated by chi-squared analysis (data not shown). These figures were further confirmed using a second method (Hu and Smyth, 2009).

Effects of Notch and Hedgehog Inhibitors on Tumor Initiation.

To analyze whether the inhibition of the Notch and Hedgehog developmental pathways could affect the tumor initiating capacity of the cancer stem cells in vivo, 100 HLA-negative sorted cells from human primary prostate tumor xenografts were inoculated subcutaneously into NSG mice. Mice were treated the same day of cell injection with vehicle solution (Control), Dexamethasone (15 mg/kg/ip daily), Cyclopamine (50 μg/kg/sc daily) plus Dexamethasone, DBZ (10 μM/kg/ip daily) plus Dexamethasone, or a combination of the 3 drugs. Dexamethasone and Cyclopamine were administered daily until the end of the experiment; DBZ was administered daily (days 1 to 15 every 4 weeks) in order to avoid gut toxicity. Four mice injected in both upper flanks were included for each treatment arm. Mice were monitored every day until tumors formed. Animals were sacrificed if they showed any evidence of distress or if they lost more than 20% of their original body weight. Generated tumors were harvested and histologically confirmed.

Statistical Analyses.

Statistical analysis was carried out with SPSS version 19.0 (SPSS, Inc.). Experimental data was expressed as means±SD and analyzed by Student's t-test. Association between the percentage of CK-negative cells and biochemical (PSA) disease recurrence was analyzed by the Kaplan-Meier method, and curves were compared by the log-rank test. All the statistical tests were conducted at the two-sided 0.05 level of significance.

### Example 2

### Docetaxel Resistant Prostate Cancer Cells Lack Differentiation Markers and Show Upregulation of the Notch and Hedgehog Signaling Pathways

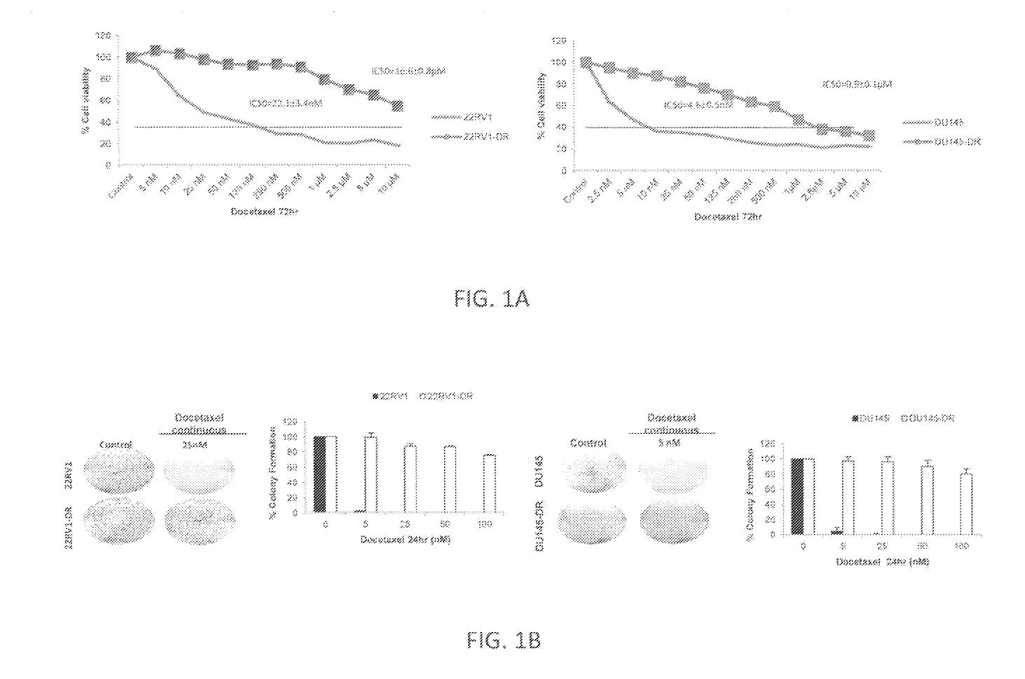

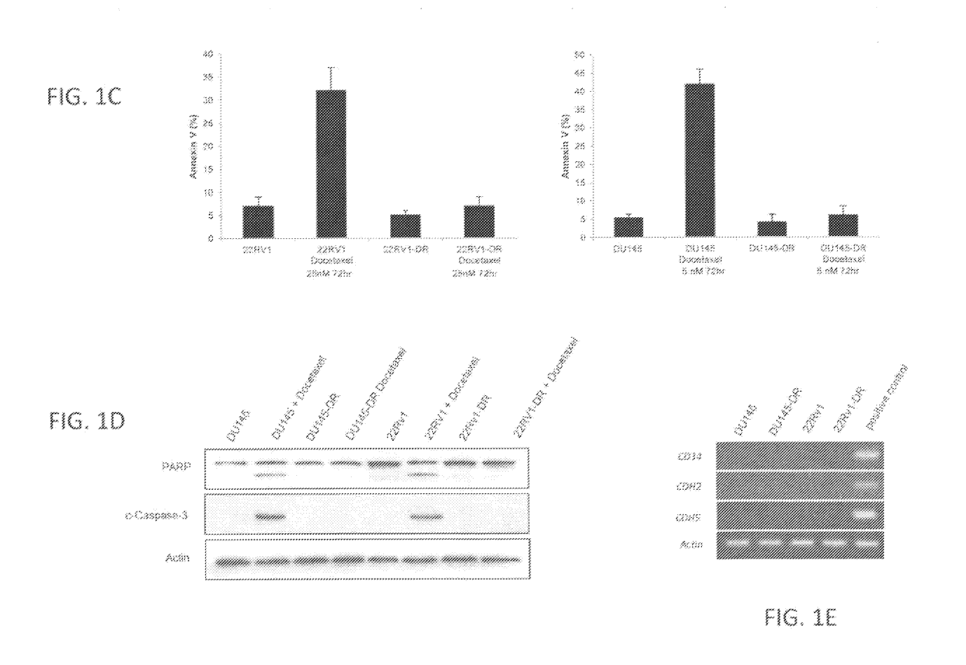

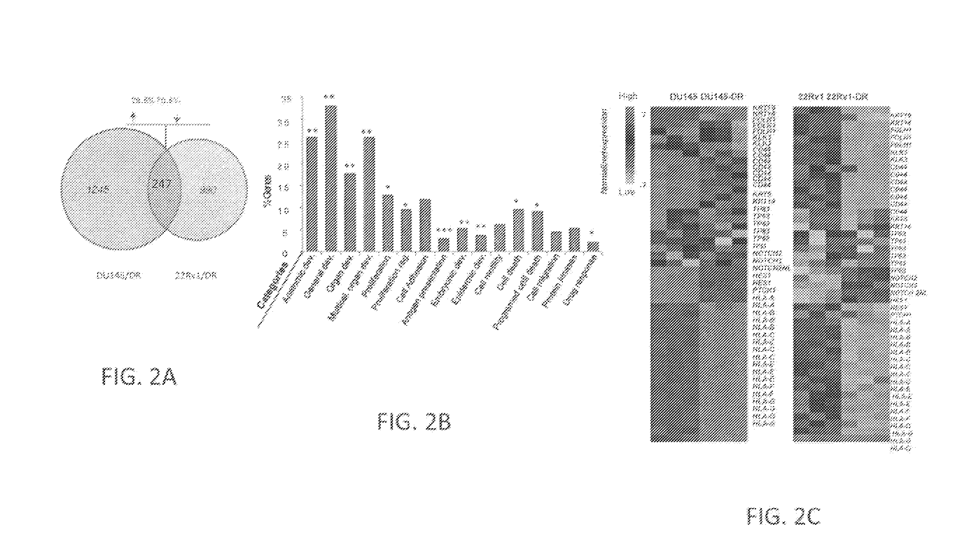

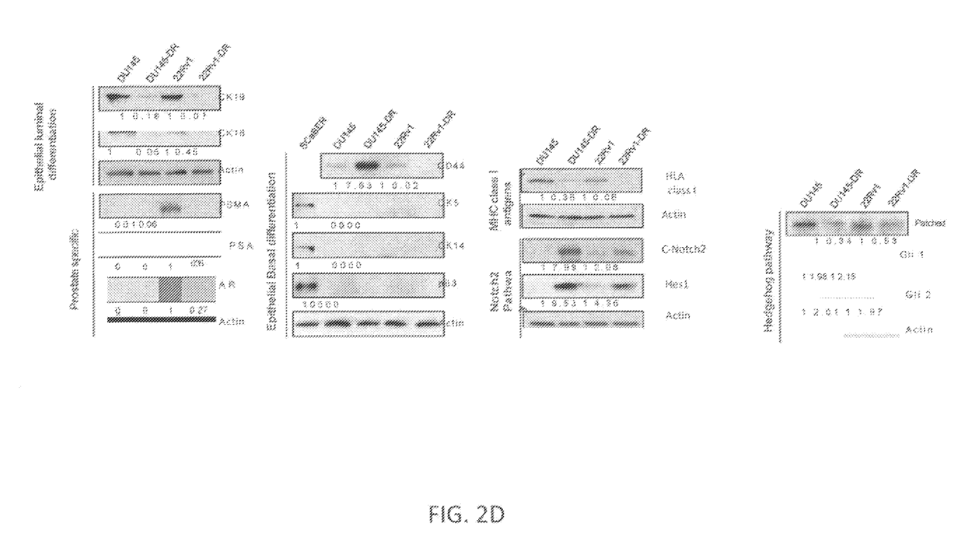

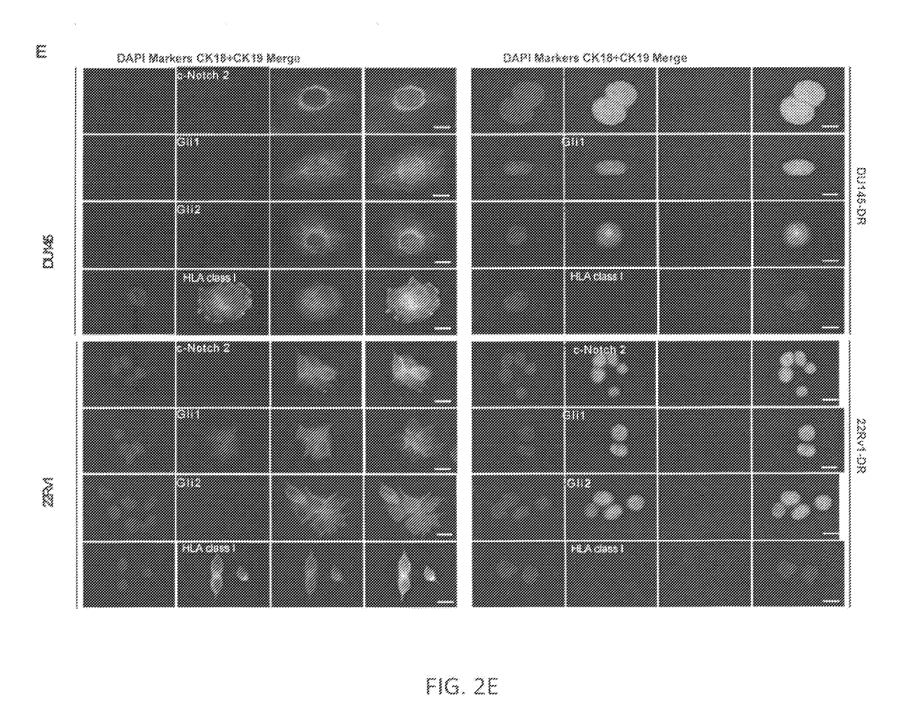

To study the phenomenon of relapse following Docetaxel therapy, chemoresistance models were generated in vitro using the well established HRPC cell lines, DU145 and 22Rv1. Drug resistant cells were established by exposure to increasing concentrations of Docetaxel, and resistance was validated by cell viability (FIG. 1A), colony formation (FIG. 1B), Annexin V (FIG. 1C), and poly-(ADP-ribose) polymerase (PARP) cleavage assays (FIG. 1D). Gene expression profiling using oligonucleotide microarrays was performed to compare the sensitive parental cells (DU145/22Rv1) with the Docetaxel resistant cells (DU145-DR/22Rv1-DR). This analysis revealed 1245 deregulated genes in DU145-DR cells and 990 deregulated genes in 22Rv1-DR cells, of which 247 overlapped (FIG. 2A). Of these overlapping genes, 29.5% were consistently upregulated and 70.5% were consistently downregulated. Gene Ontology (GO) analysis of these 247 genes revealed that, in addition to expected changes in biological processes such as cell proliferation, cell death, and drug response, other categories including cell differentiation, antigen presentation, and developmental/stemness pathways were significantly represented (FIG. 2B).

cDNA microarray analysis revealed that Docetaxel resistant (DU145-DR and 22Rv1-DR) cells had reduced epithelial differentiation, prostate specific, and HLA class I gene signatures when compared to parental (DU145 and 22Rv1) cells (FIG. 2C). Additionally, Docetaxel resistant (DU145-DR and 22Rv1-DR) cells had increased developmental (Notch and Hedgehog) gene signatures when compared to parental (DU145 and 22Rv1) cells (FIG. 2C). Regarding differentiation, expression of the low molecular weight cytokeratins (CKs) 18 and 19 was analyzed, as these epithelial markers are specifically expressed in normal luminal human prostate cells and prostate cancer (Ali and Epstein, 2008). Prostate related biomarkers, including the androgen receptor (AR), prostate specific antigen (PSA), and prostate specific membrane antigen (PSMA), were also analyzed. DU145-DR and 22Rv1-DR cells showed a dramatic decrease in gene transcription (FIG. 2C) and protein expression levels of CKs 18 and 19 (FIGS. 2D-E). 22Rv1 cells, which express prostate-related differentiation markers, showed a decrease in gene and protein expression levels of PSMA and PSA, as well as a decrease in AR protein expression in Docetaxel resistant cells (FIG. 2D). Because loss of luminal markers could indicate a possible shift to a basal phenotype, the expression of high molecular weight CKs and the prostate basal markers, CD44 and p63, were also analyzed. High molecular weight CKs (CKs 5 and 14) and p63 remained undetectable in the drug resistant cells as well as in their respective parental cells (FIGS. 2C and D). CD44 transcription and protein expression were increased in DU145-DR and decreased in 22RV1-DR relative to their parental lines, indicating a cell line dependent effect (FIGS. 2C and D). Therefore, the decrease in luminal differentiation and prostate specific markers was not associated with a consistent shift to a basal phenotype. Further, Docetaxel resistant cells did not express other lineage markers (FIG. 1E). Lineage markers were negative in both sensitive and Docetaxel resistant DU145 and 22Rv1 cells when compared to appropriate positive controls. Positive controls were human hematopoietic stem cells obtained from cord blood (hematopoietic lineage), NIH3T3 cells (mesenchyme lineage), and HUVEC cells (endothelial lineage). Finally, Docetaxel resistant cells showed a strong downregulation of the gene transcription levels of HLA class I antigens A, B, C, E, F, and G (FIG. 2C), which was further confirmed with a pan-HLA class I antibody by immunoblotting (FIG. 2D) and immunofluorescence (FIG. 2E).

Regarding the developmental/stemness category, Docetaxel resistant cells showed a marked upregulation of the Notch and Hedgehog signaling pathways. There was increased NOTCH2 and HES1 gene transcription levels (FIG. 2C), which was associated with increased cleaved Notch2 and Hes1 protein expression (FIG. 2D) and cleaved Notch2 localization within the nucleus, where it exerts its activity (FIG. 2E). Moreover, resistant cells showed reduced expression of the Hedgehog receptor Patched, which normally inhibits the activity of Smo, a positive regulator of the Hedgehog pathway (FIGS. 2C and D). This was associated with increased protein levels and nuclear localization of the transcription factors Gli1 and Gli2 (FIGS. 2D and E), consistent with Hedgehog pathway activation. In summary, Docetaxel resistant HRPC cells displayed a phenotype characterized by loss of epithelial differentiation markers, prostate specific antigens, and antigen presentation molecules, as well as an increase in the Notch and Hedgehog developmental signaling pathways.

### Example 3

### Primary and Metastatic Prostate Cancer Tissues Contain Cells that Display the Docetaxel Resistance Phenotype and Associate with Tumor Aggressiveness

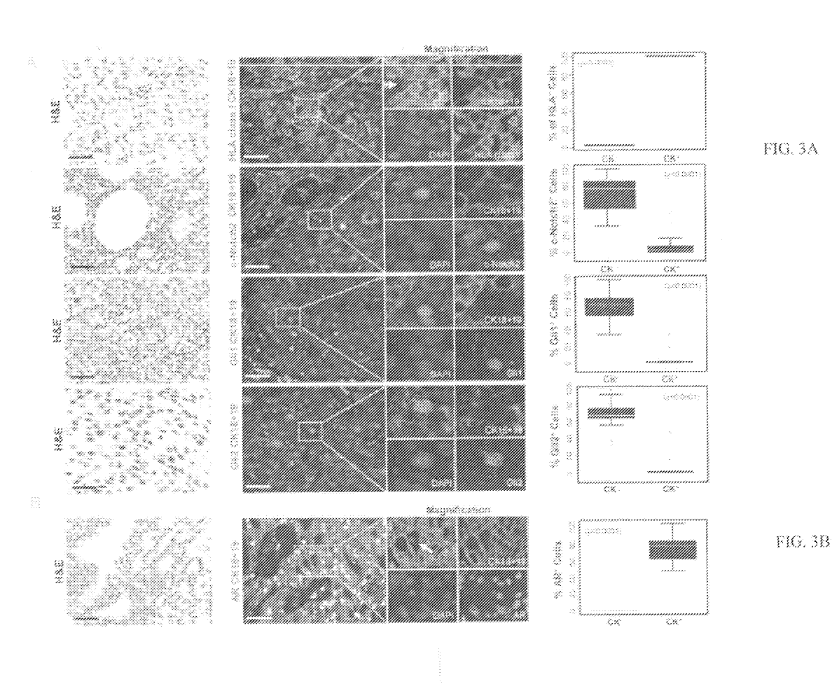

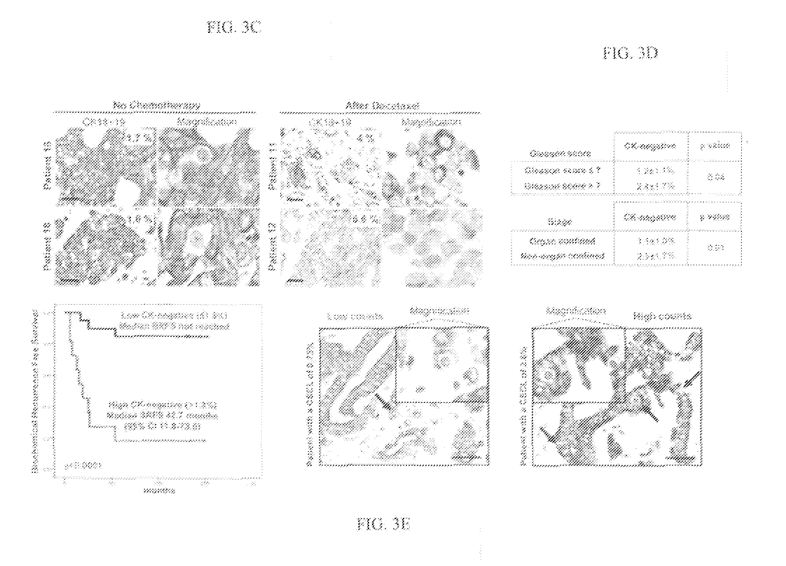

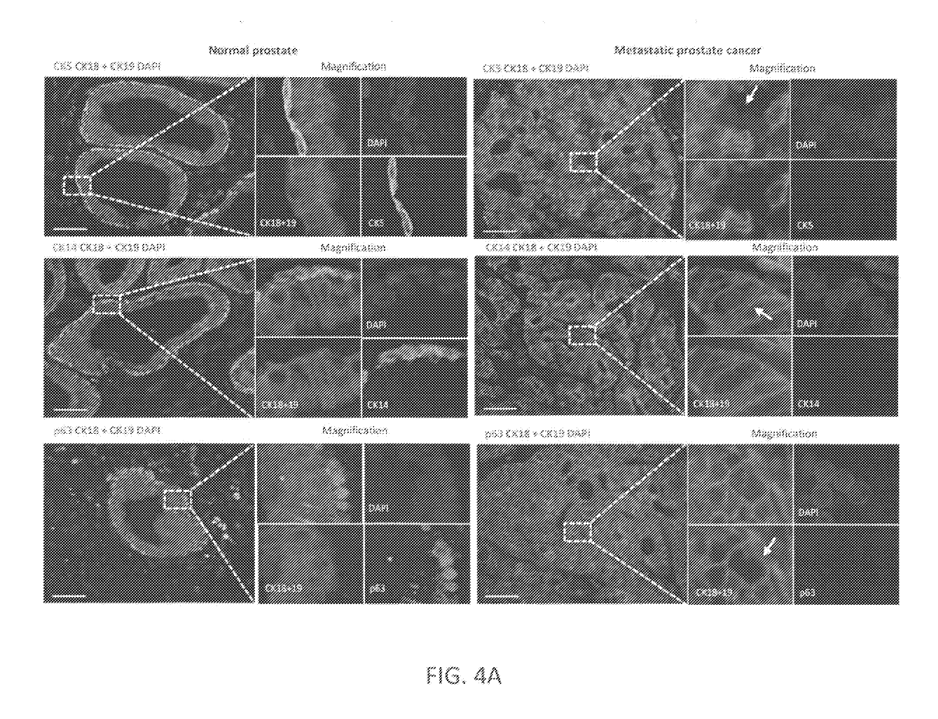

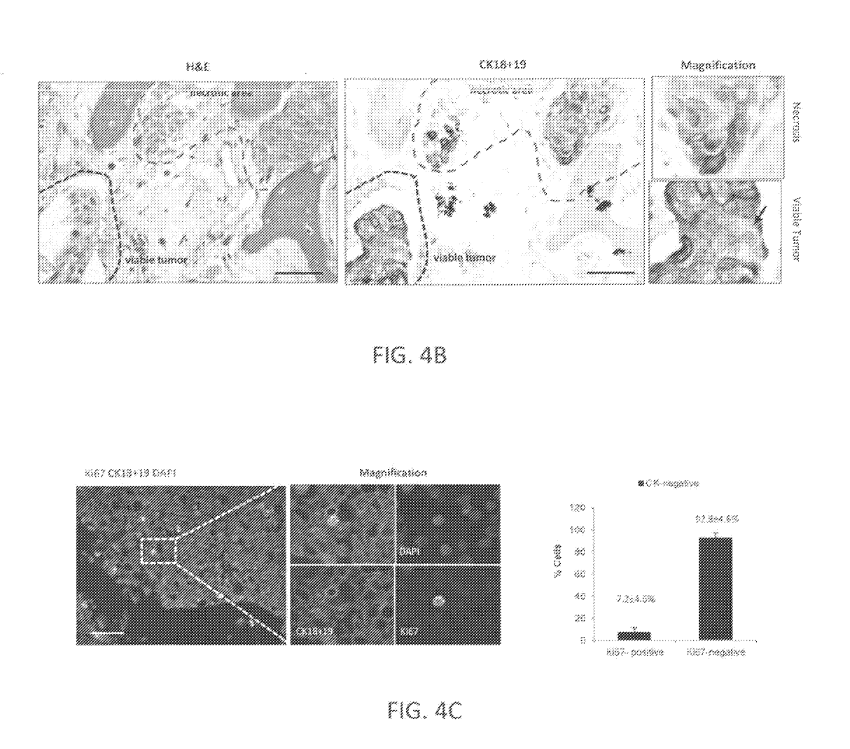

Experiments were conducted to determine whether cells with the identified Docetaxel resistant phenotype were detectable in human prostate cancer tissue samples. Analysis was carried out on paraffin embedded tissues from 31 untreated primary prostate tumors from patients who had undergone radical prostatectomy and 36 metastatic prostate cancer tissue samples from untreated or Docetaxel treated patients. Immunofluorescence-based double staining revealed that all prostate cancer tumors had a small subpopulation of CK-negative tumor cells that displayed the Docetaxel resistant phenotype observed in the in vitro models described in Example 2. CK18 and CK19-negative cells were mainly HLA class I-negative (98.5±1.1%) and displayed nuclear expression of cleaved Notch2 (72.8±15.1%), Gli1 (67.5±17.3%), and Gli2 (67±17.3%), whereas CK-positive cells were HLA class I-positive (99.6±0.3%) and showed significantly lower nuclear expression of developmental transcription factors (p<0.0001, FIG. 3A). Moreover, CK-negative tumor cells lacked expression of nuclear AR, whereas CK-positive cells displayed nuclear AR in 71.8±14.3% of the cells (p<0.0001, FIG. 3B). Further, CK-negative cells did not express high molecular weight CKs 5 and 14, or p63 (FIG. 4A). Finally, CK-negative cells did not exhibit morphological criteria of necrosis (FIG. 4B), and a subset expressed the proliferative marker Ki67 (FIG. 4C).

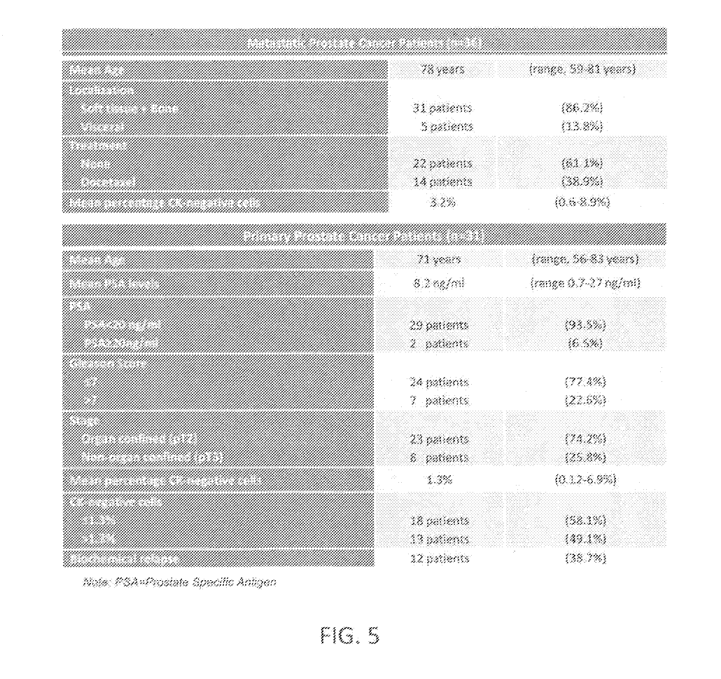

Quantitative analysis revealed that in 31 primary tumors, CK-negative cells accounted for a mean of 1.3±0.94% of the total tumor cell population, whereas in the 36 metastatic prostate tissues, this cell population accounted for 3.2±2.2% (FIG. 5). Therefore, specimens from advanced disease exhibited a higher percentage of CK-negative cells (p<0.0001). Notably, 14 out of the 36 metastatic prostate cancer samples analyzed belonged to patients who had been previously treated with Docetaxel and had the highest percentage of CK-negative cells (5.2±2.1%), whereas the other 22 non-treated patients had a lower percentage of CK-negative cells (1.8±1.4%; p<0.0001; FIG. 3C). Thus, cells with the Docetaxel resistant phenotype were more abundant in metastatic tumors and after chemotherapy treatment.

Next, experiments were performed to investigate if this subpopulation had prognostic significance in primary prostate cancer. Quantitative analysis performed in the 31 primary prostate tumor samples showed that the percentage of the CK-negative cells was significantly related to established clinico-pathologic prognostic factors like tumor grade (Gleason score) and pathological disease stage (FIG. 3D). Further, the percentage of CK-negative cells was linked to the risk of disease relapse (FIG. 3E). Patients with a high percentage of CK-negative cells (>1.3%) had a median time to biochemical (PSA) relapse of 42.7 months (95% CI 11.8-73.6 months), whereas patients with a low percentage of CK-negative cells (≦1.3%) did not reach median time to biochemical relapse (p<0.0001). In summary, cells displaying the Docetaxel resistance phenotype were detectable in primary and metastatic prostate cancer tissue samples, increased in number after chemotherapy, and their abundance was associated with tumor aggressiveness and clinical prognosis.

### Example 4

### A Subpopulation of Prostate Cancer Cells Exhibits the Docetaxel Resistance Phenotype and Survives Docetaxel Exposure

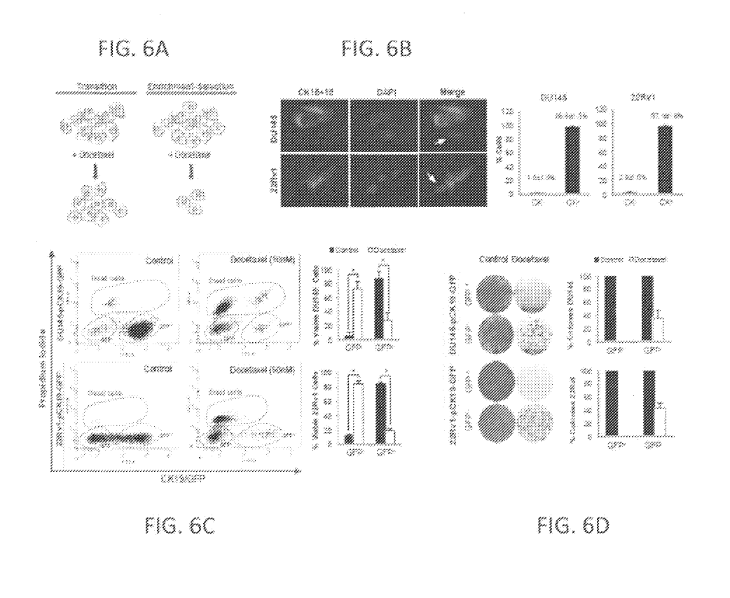

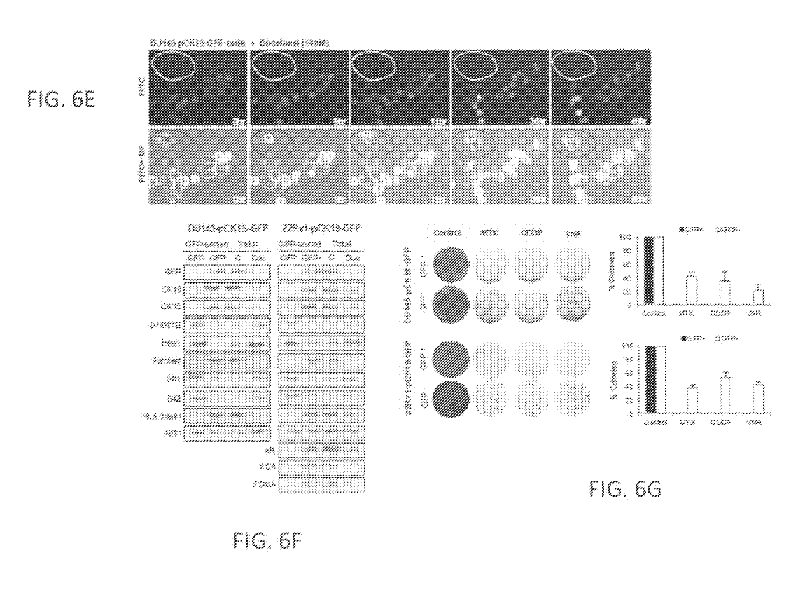

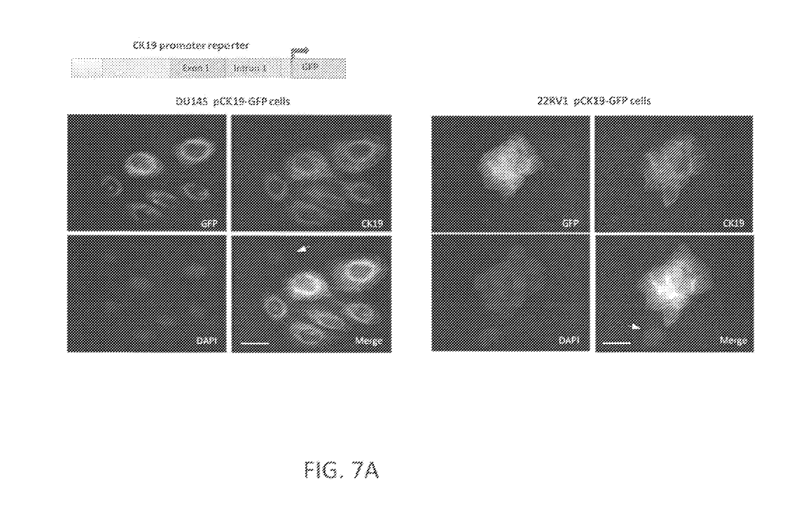

Having characterized DU145-DR and 22Rv1-DR cells in vitro and identified the Docetaxel resistance phenotype in a subpopulation of tumor cells in clinical samples, the next set of experiments sought to investigate whether the changes observed during the acquisition of Docetaxel resistance were the result of transition of sensitive cells toward a resistant phenotype, or if chemotherapy had selected for a subpopulation of intrinsically Docetaxel resistant cells (FIG. 6A). Since both DU145-DR and 22Rv1-DR cells displayed down-regulation of CK19 and CK18, these markers were chosen to determine if CK-negative cells were present in the parental cell lines before any treatment. Immunofluorescence and flow cytometry revealed a small CK-negative subpopulation representing 1.8±1.5% and 2.9±1.6% of DU145 and 22Rv1 parental cells, respectively (FIG. 6B).

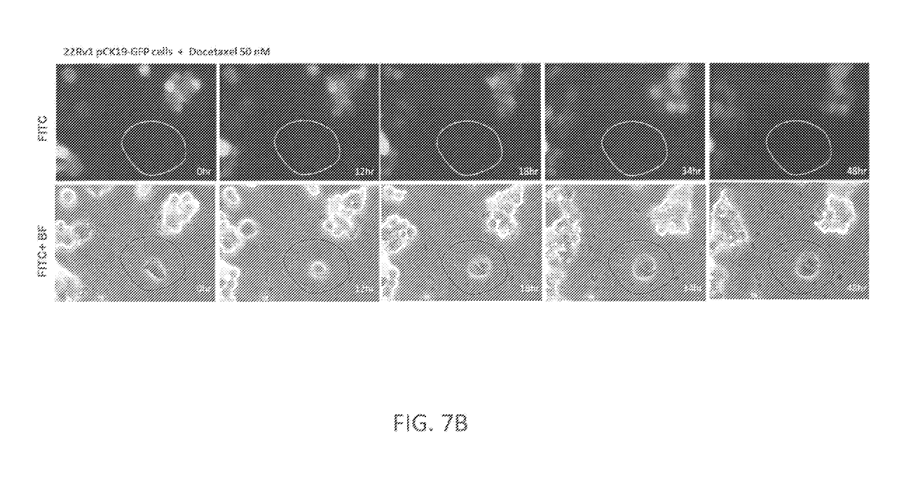

Experiments were carried out to investigate if this subpopulation could contribute to the acquisition of Docetaxel resistance. To test this hypothesis, a strategy was designed to track the behavior of CK-negative cells under chemotherapy. A region of the CK19 promoter was cloned into a GFP vector, creating a reporter system for the expression of CK19 under different experimental conditions (Tripathi et al., 2005). DU145 and 22Rv1 parental cells were transfected with the pCK19-GFP construct and selected to establish stable cell lines, named DU145-pCK19-GFP and 22Rv1-pCK19-GFP, respectively. Co-expression of CK19 and GFP was validated by immunofluorescence (FIG. 7A), and PCR confirmed stable integration of the construct (data not shown).

Then, experiments were performed to test whether CK19/GFP-negative cells survived Docetaxel exposure and were responsible for acquired chemoresistance (FIG. 6C). Analysis of DU145-pCK19-GFP cells by flow cytometry showed that CK19/GFP-positive cells were a majority (87.5±10.4%) of the total population before any treatment. However, the viable CK19/GFP-positive population was reduced to 28.3±10.6% after exposure to 10 nM Docetaxel for 72 h (p<0.0001). In contrast, the viable CK19/GFP-negative population increased proportionally from 4.4±4.3% to 73.1±10.2%, indicating an enrichment after chemotherapy (p<0.0001). Similar results were observed in 22Rv1-pCK19-GFP cells after exposure to 50 nM Docetaxel. Viable 22Rv1-pCK19-GFP-negative cells increased proportionally from 8.5±3.5% to 78.6±4.1%, while viable CK19/GFP-positive cells decreased from 85.0±2.0% to 19.3±3.4% (p<0.0001). Further, colony formation assays showed that only CK19/GFP-negative cells formed colonies after exposure to Docetaxel (FIG. 6D). Next, the behavior of DU145-pCK19-GFP and 22Rv1-pCK19-GFP cells in the presence of Docetaxel was analyzed by live imaging. CK19/GFP-negative cells were able to divide and exit mitosis under therapy, whereas CK19/GFP-positive cells died after mitotic arrest (FIGS. 6E and 7B). Finally, the CK19/GFP-negative and CK19/GFP-positive populations of DU145-pCK19-GFP and 22Rv1-pCK19-GFP cells were characterized under both control and Docetaxel-treated conditions (FIG. 6F). Immunoblots confirmed that CK19/GFP-negative cells exhibited the Docetaxel resistance markers, namely reduced CK18, CK19, HLA class I, Patched, AR, PSMA, and PSA expression, as well as upregulation of cleaved Notch 2, Hes1, Gli1, and Gli2. Moreover, unsorted cells treated with Docetaxel underwent the expected reduction in differentiation markers and increase in developmental signaling pathways.

Additionally, CK19/GFP-negative cells exhibited a multi-drug resistance phenotype. CK19/GFP-negative cells from DU145-pCK19-GFP and 22Rv1-pCK19-GFP treated with DNA damaging agents (Mitoxantrone and Cisplatin) and other anti-mitotic agents (Vinorelbine) formed colonies, but CK19/GFP-positive cells failed to do so (FIG. 6G).

### Example 5

### Combined Notch and Hedgehog Signaling Inhibition Depletes Docetaxel Resistant Prostate Cancer Cells

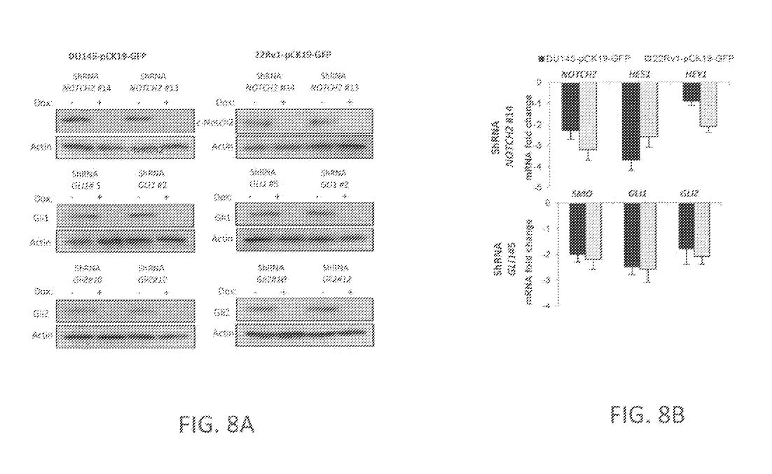

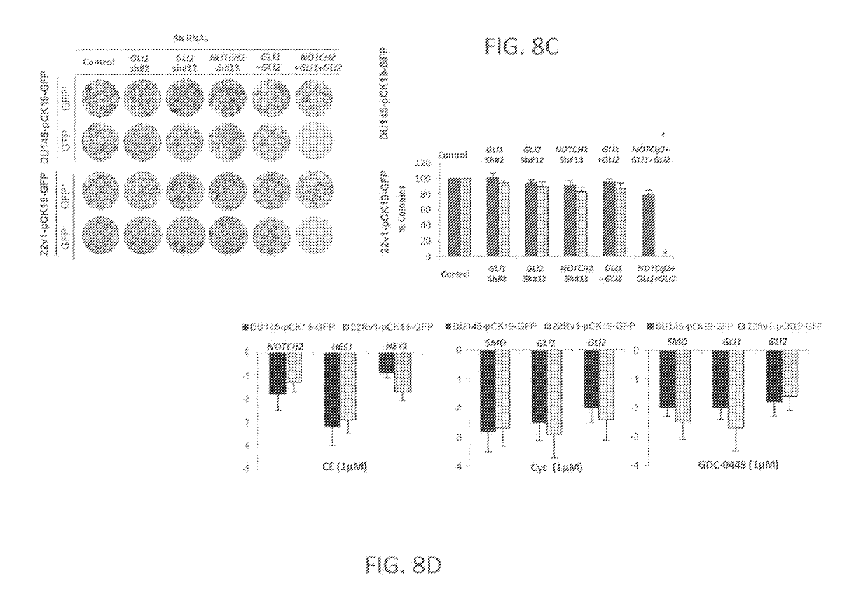

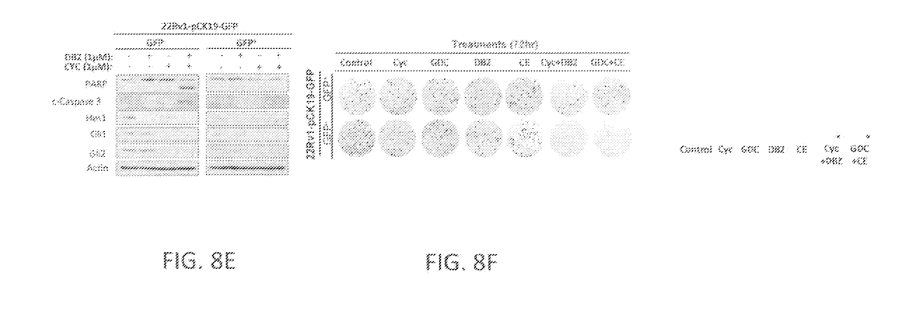

Given the findings that CK19-negative cells mediated acquired Docetaxel resistance in vitro, and that these cells were more abundant in prostate cancer patients treated with Docetaxel, experiments were performed to investigate whether these cells could be targeted to inhibit acquired resistance to Docetaxel. The upregulation of Notch and Hedgehog signaling in DU145-DR and 22Rv1-DR cells (FIG. 2) prompted the investigation of the role of these pathways in the survival of CK-negative cells. RNAi silencing was used to knock down genes critical for Notch and Hedgehog signaling in DU145-pCK19-GFP and 22Rv1-pCK19-GFP cells. ShRNAs against NOTCH2, GLI1, and GLI2 were used in biological replicates conferring a 90% reduction in protein levels (FIG. 8A). NOTCH2 knockdown reduced the mRNA levels of the Notch target genes HES1 and HEY1, and GLI1 and GLI2 knockdown reduced the mRNA levels of the Hedgehog target gene SMO, confirming that knockdown of these genes disrupted Notch and Hedgehog signaling (FIG. 8B).

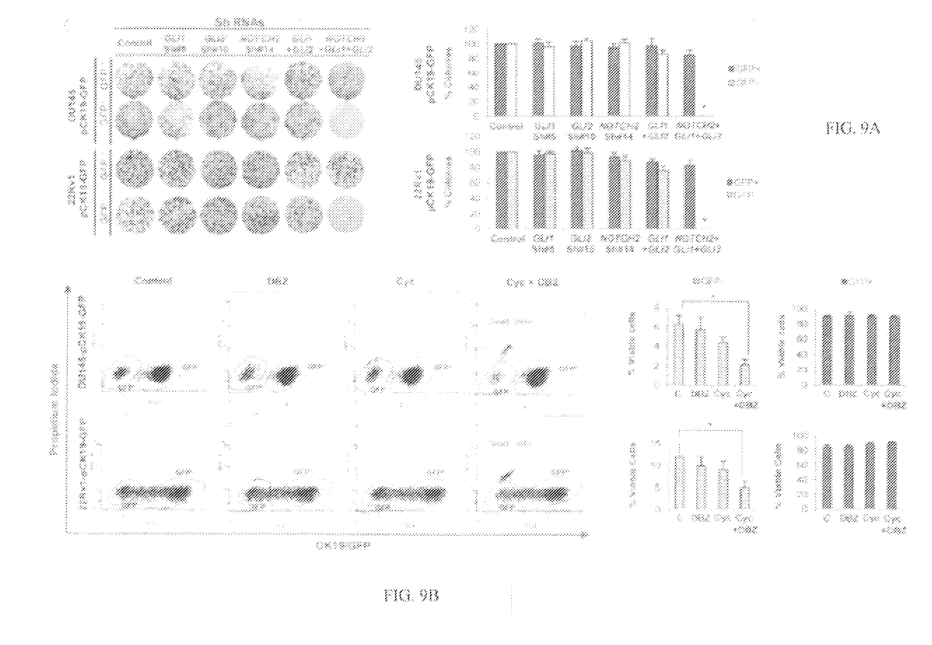

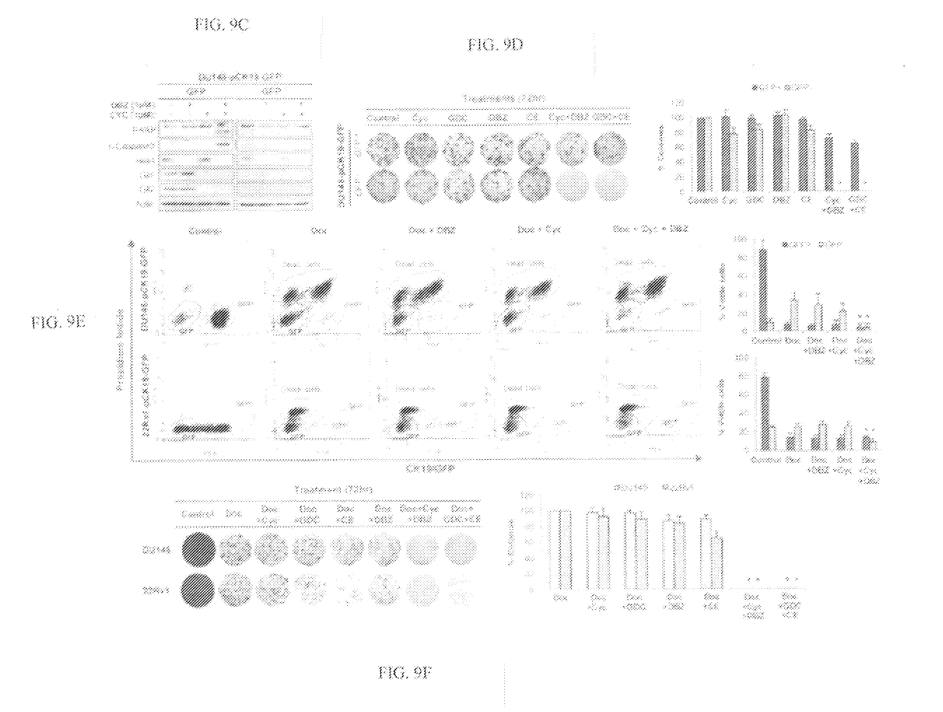

The effects of Notch and Hedgehog knockdown on CK19-negative cells were then analyzed. Colony formation assays of GFP-negative and GFP-positive sorted DU145-pCK19-GFP and 22Rv1-pCK19-GFP cells expressing shRNAs against NOTCH2, GLI1, and GLI2 revealed that individual knockdown of Notch or Hedgehog signaling did not have an effect on the colony formation of CK19/GFP-negative or CK19/GFP-positive cells (FIGS. 9A and 8C). In contrast, concomitant knockdown of both pathways dramatically abrogated the ability of CK19/GFP-negative cells to form colonies, while CK19/GFP-positive cells were unaffected (FIGS. 9A and 8C). These results indicate that both Notch and Hedgehog signaling pathways in combination are required for the maintenance of cells displaying the Docetaxel resistant phenotype.

These findings were further validated using the chemical inhibitors, Cyclopamine and GDC-0449, Hedgehog pathway antagonists that act at the level of Smo (Chen et al., 2002; Karhadkar et al., 2004; Robarge et al., 2009; Taipale et al., 2000), and DBZ and Compound E, gamma-secretase inhibitors that block proteolytic processing of Notch (Seiffert et al., 2000; van Es et al., 2005). Quantitative RT-PCR of pathway target genes confirmed that these pharmacological inhibitors were targeting their respective pathways (FIG. 8D). Flow cytometry analysis of DU145-pCK19-GFP and 22Rv1-pCK19-GFP cells treated with a combination of Notch and Hedgehog inhibitors showed a significant loss in cell viability of CK19/GFP-negative cells, and no effect on CK19/GFP-positive cells (FIG. 9B). The decrease in CK19/GFP-negative cell viability was due to the induction of an apoptotic response, as demonstrated by Caspase-3 and PARP cleavage (FIGS. 9C and 8E). Finally, as observed in the genetic knockdown studies, colony formation assays confirmed that combined pharmacological inhibition of Notch and Hedgehog signaling selectively depleted CK19/GFP-negative cells (FIGS. 9D and 8F).

These results indicated that combined Notch and Hedgehog inhibition could target CK19-negative Docetaxel resistant cells. Therefore, it was hypothesized that a combination strategy of Docetaxel plus developmental pathway inhibitors could ablate both CK-positive and CK-negative compartments, respectively. Indeed, flow cytometry analysis of DU145-pCK19-GFP and 22Rv1-pCK19-GFP cells revealed that triple combinations of Cyclopamine and DBZ with Docetaxel reduced the viability of both CK19/GFP-negative and CK19/GFP-positive cells (FIG. 9E). Triple combination completely ablated the colony formation capacity of both DU145 and 22Rv1 parental cell lines (FIG. 9F).

### Example 6

### Abrogation of Acquired Docetaxel Resistance In Vivo Through Notch and Hedgehog Signaling Inhibition

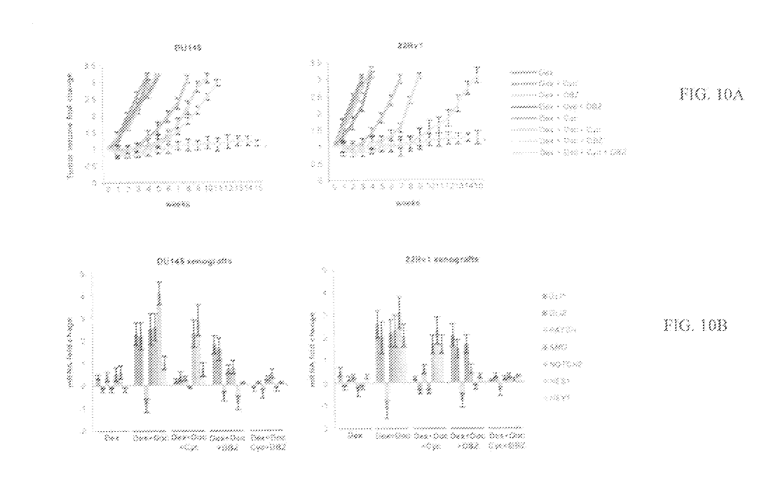

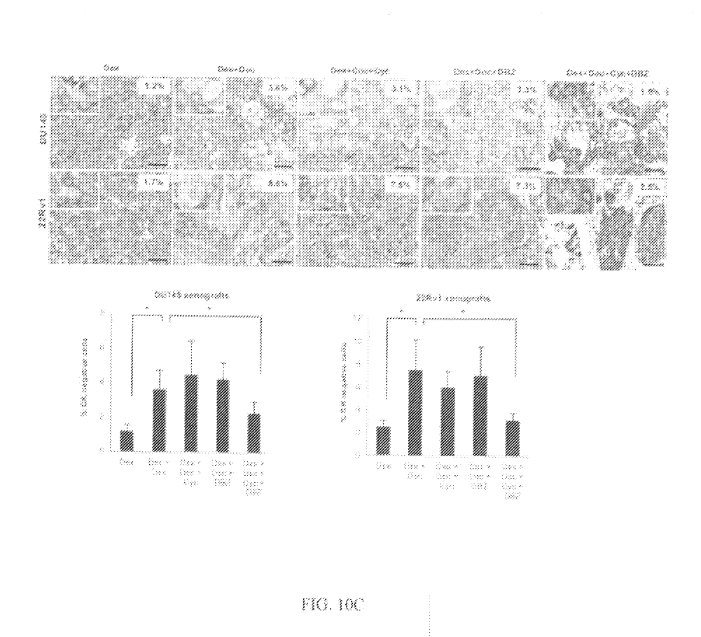

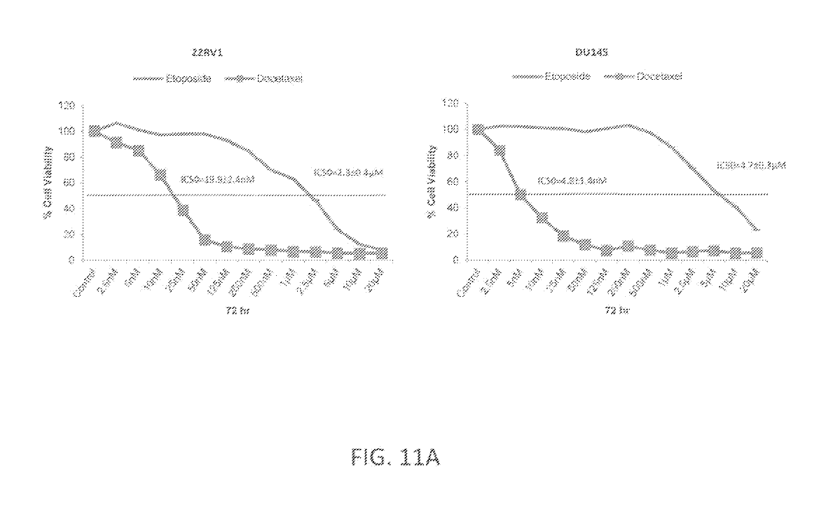

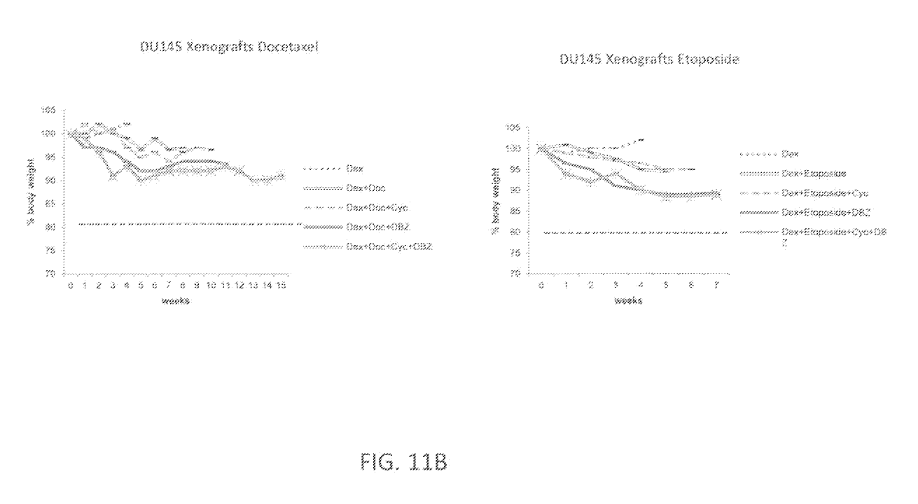

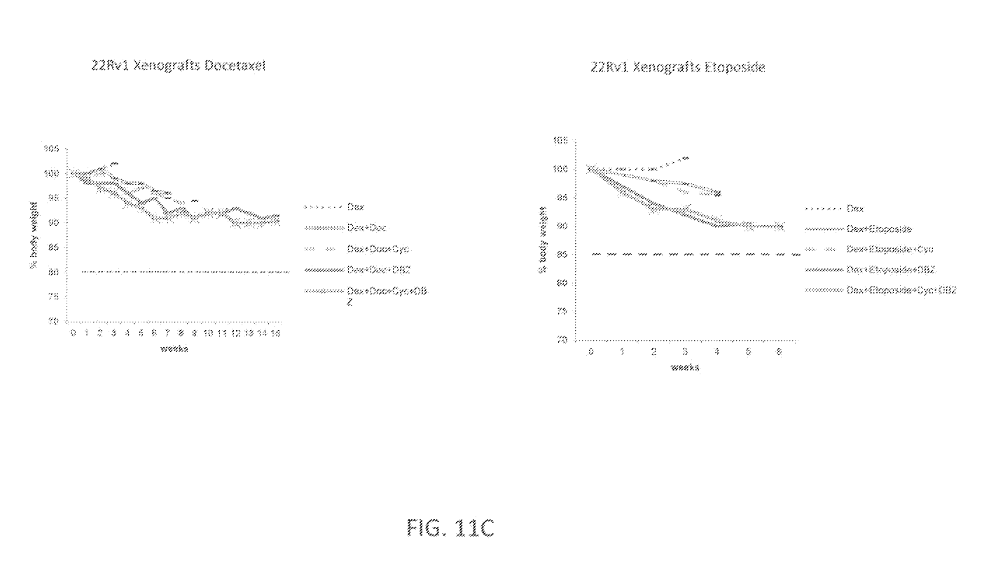

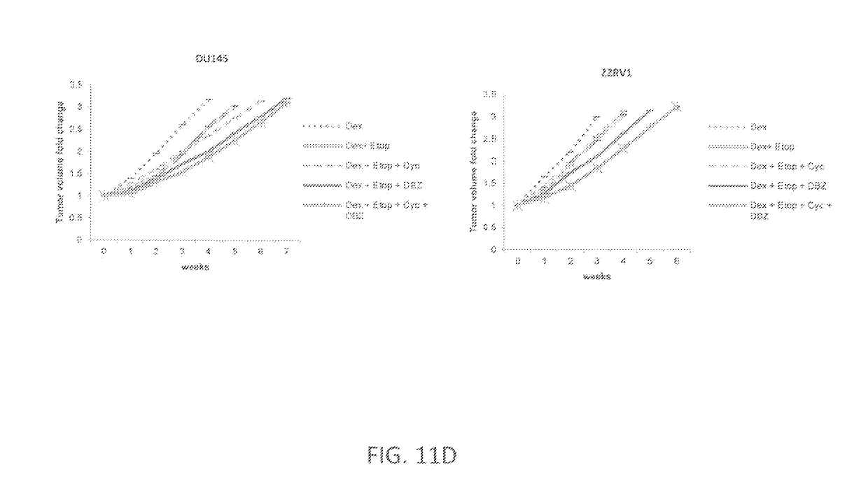

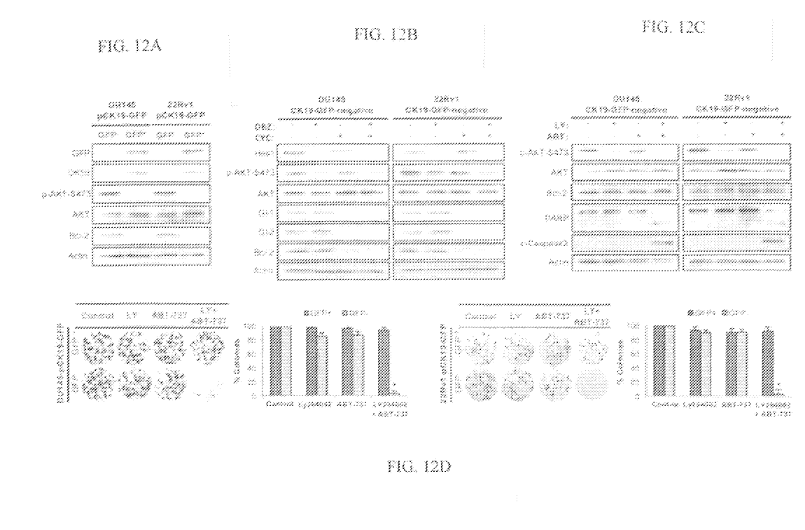

The effects of this combination strategy were then evaluated in vivo. For these experiments, Dexamethasone was used as a co-adjuvant therapy to reduce the gut toxicity of gamma-secretase inhibitors (Real et al., 2009). NOD/SCID mice bearing DU145 and 22Rv1 xenografts were treated with Dexamethasone alone, dual combinations (e.g. Dexamethasone plus Docetaxel), triple combinations (e.g. Dexamethasone plus Docetaxel plus DBZ) or a quadruple combination (Dexamethasone plus Docetaxel plus Cyclopamine plus DBZ). Xenografts treated with Dexamethasone and Docetaxel temporarily stabilized tumor volume before progression. Remarkably, mice treated with the quadruple combination showed a robust inhibition of tumor growth during the course of the experiment (15 weeks), compared to mice under the other combination regimes, therefore mirroring the in vitro results (FIG. 10A) Inhibitory effects of the drugs on their respective signaling pathways was confirmed by testing the mRNA levels of Notch and Hedgehog pathway genes in tumor cells obtained from xenografts 4 hours after drug administration (FIG. 10B). Moreover, in agreement with the in vitro and human sample results in the previous examples, Docetaxel treatment of DU145 and 22Rv1 xenografts enriched for CK-negative cells, and xenografts treated with the quadruple combination displayed a lower percentage of CK-negative cells in comparison to Docetaxel treated animals (FIG. 10C). To control for possible drug toxicity associated with the quadruple combination, Docetaxel was substituted with Etoposide, a chemotherapy agent with minor efficacy in prostate cancer (FIG. 11A). Whereas similar toxicity (% body weight reduction) was observed (FIG. 11B) as when Docetaxel was used, there was no significant delay in tumor growth (FIG. 11C), indicating that the efficacy of the quadruple therapy was not a result of drug toxicity. These results indicated that CK-negative Docetaxel resistant cells were critically dependent on Notch and Hedgehog signaling, giving the rationale for an efficacious combination strategy.

### Example 7

### Notch and Hedgehog Signaling Regulate Survival Molecules in Docetaxel Resistant Cells

The results from the studies in Examples 2-6 suggested that Notch and Hedgehog signaling were critical regulators of acquired Docetaxel resistance (FIGS. 9 and 10). In order to elucidate the molecular mechanisms underlying these observations, the downstream effectors of Notch and Hedgehog signaling in CK19/GFP-negative Docetaxel resistant cells were investigated. Specifically, experiments were carried out to test whether Notch and Hedgehog signaling may regulate CK19/GFP-negative Docetaxel resistant cells through pro-survival and anti-apoptotic mechanisms, respectively.

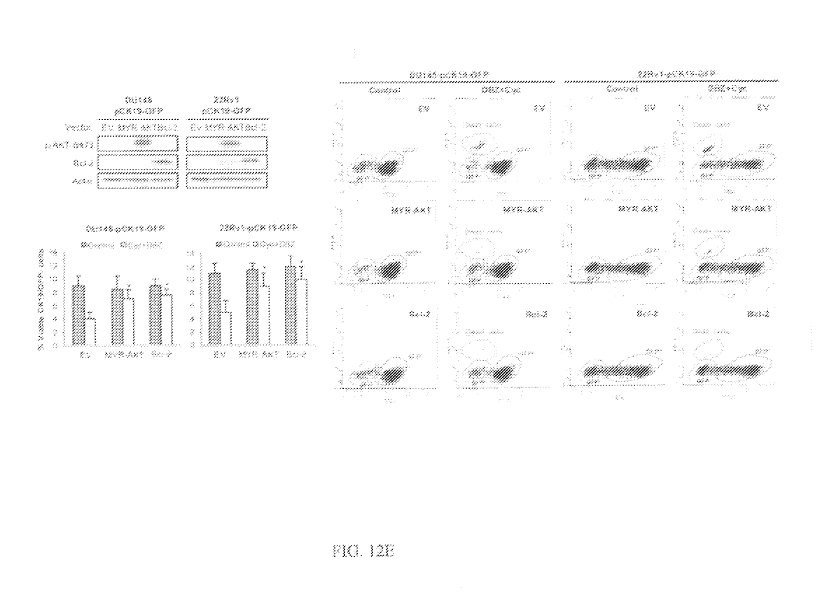

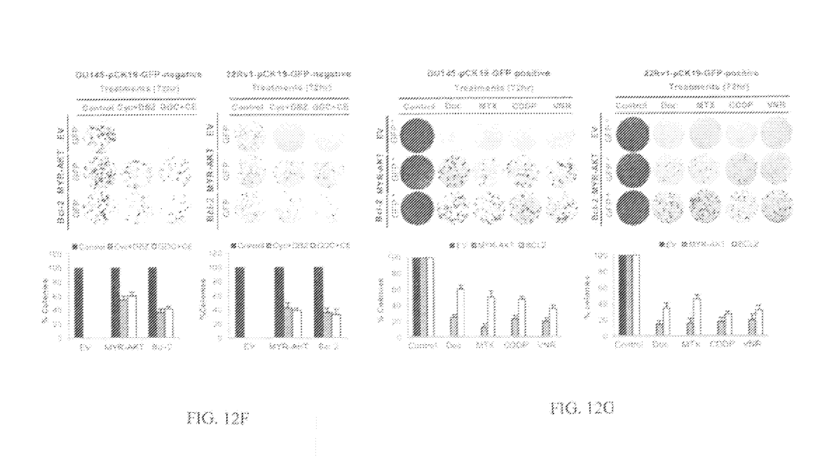

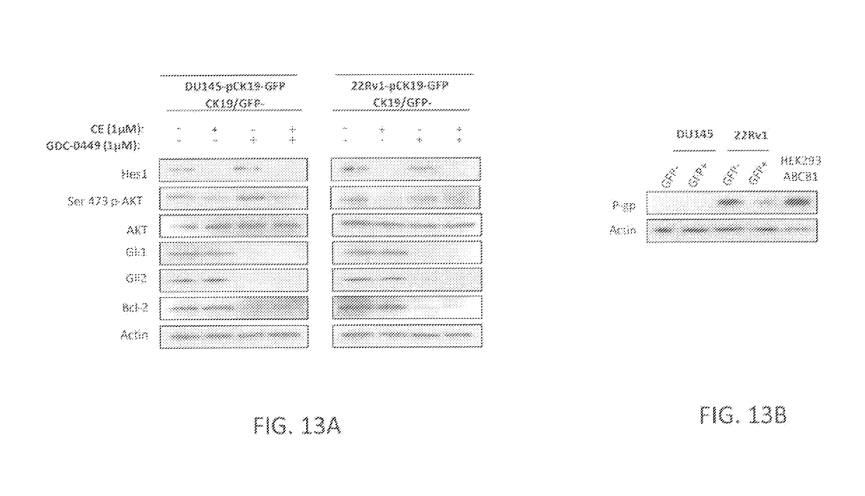

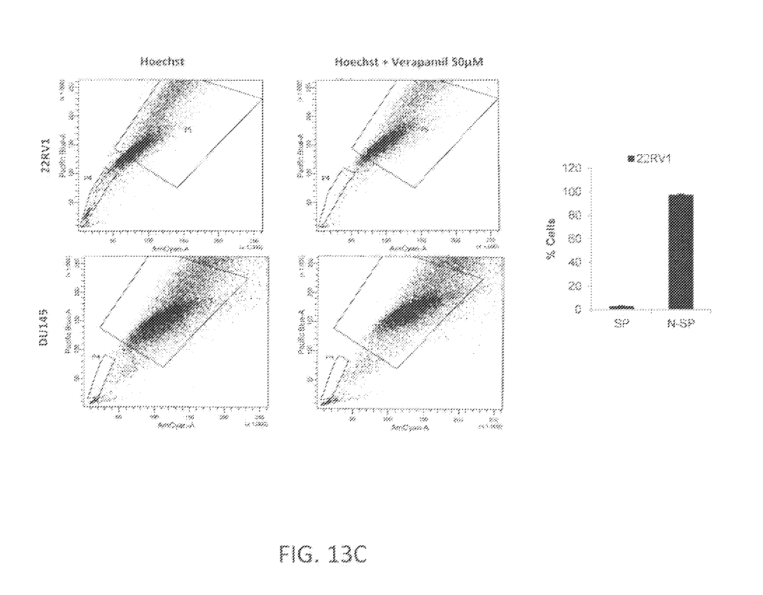

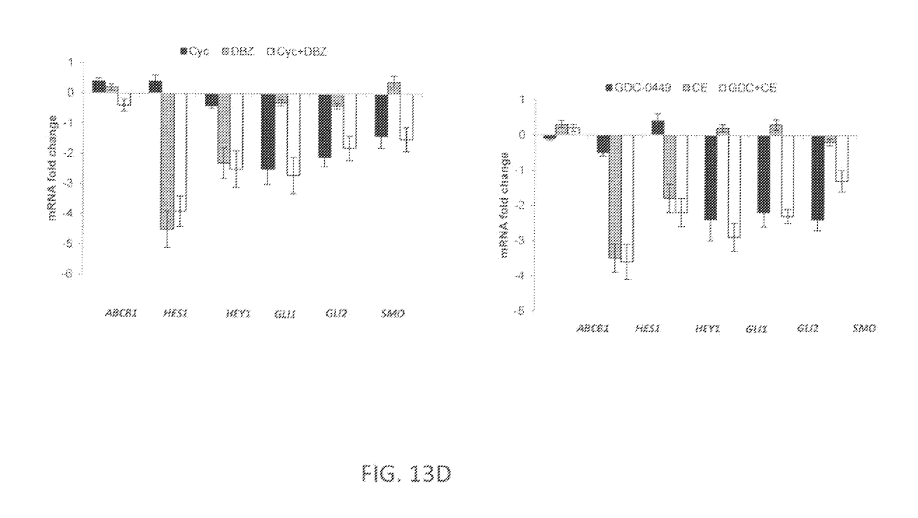

First, AKT phosphorylation (Ser473) and Bcl-2 expression in the CK19/GFP-negative and CK19/GFP-positive populations of DU145-pCK19-GFP and 22Rv1-pCK19-GFP cells were examined. Immunoblots revealed that, in both cell lines, the CK19/GFP-negative compartment displayed increased levels of p-AKT (Ser473) and Bcl-2 (FIG. 12A). To assess whether these survival molecules were indeed regulated by Notch and Hedgehog signaling in CK19/GFP-negative cells, experiments were performed using chemical inhibitors of Notch (DBZ and CE) and Hedgehog (Cyclopamine and GDC-0449) signaling. Immunoblots showed that inhibition of Notch signaling with either DBZ or CE and inhibition of Hedgehog signaling with either Cyclopamine or GDC-0449 significantly reduced p-AKT (Ser473) and Bcl-2 expression levels, respectively (FIGS. 12B and 13A).

To determine whether the activity of these downstream effectors is necessary for CK19/GFP-negative cell survival, the combined effects of LY294002, a selective inhibitor of the PI3K/AKT pathway (Vlahos et al., 1994), and ABT-737, an inhibitor of the Bcl-2 family members (Oltersdorf et al., 2005), were tested. These studies revealed that, combined, but not individual, PI3K/AKT and Bcl-2 inhibition induced apoptosis (FIG. 12C) and reduced colony formation (FIG. 12D) in CK19/GFP-negative cells, recapitulating the effect observed with Notch and Hedgehog inhibitors (FIG. 9). To further validate the role of Notch and Hedgehog signaling in CK19/GFP-negative cells through PI3K/AKT and Bcl-2, rescue experiments were performed using overexpression vectors of BCL2 and a constitutively active myristoylated form of AKT (MYR-AKT). Combined inhibition of Notch and Hedgehog signaling reduced the viability and colony formation of CK19/GFP-negative cells, while overexpression of either MYR-AKT or Bcl-2 reduced the inhibitory effect of these inhibitors (FIGS. 12E-F). Interestingly, overexpression of either MYR-AKT or Bcl-2 in the CK19/GFP-positive populations of DU145-pCK19-GFP and 22Rv1-pCK19-GFP cells was sufficient to confer a multi-drug (Docetaxel, Mitoxantrone, Cisplatin, and Vinorelbine) resistant phenotype in these previously sensitive cells (FIG. 12G).

Next, the expression of drug efflux mechanisms in CK19/GFP-negative cells was assessed. Taxane chemotherapeutics, among others, have been suggested to be substrates for p-Glycoprotein/ABCB1 (Gottesman et al., 2002). In these studies, P-gp/ABCB1 was elevated in the CK19/GFP-negative population of 22Rv1-pCK19-GFP cells, but not DU145-pCK19-GFP cells (FIG. 13B). Consistent with these results, 22Rv1, but not DU145 cells, exhibited a side population using a Hoechst 33342 assay (FIG. 13C). Finally, Notch and Hedgehog signaling did not regulate ABCB1 transcriptional levels in 22Rv1-pCK19-GFP-negative cells (FIG. 13D). Thus, expression of this drug efflux molecule was not a consistent feature of CK19/GFP-negative cells. Taken together, these data suggest that Notch and Hedgehog signaling regulate the activation of AKT and expression of Bcl-2, respectively, to promote survival and multidrug resistance in a P-gp/ABCB1 drug efflux independent mechanism.

### Example 8

### Docetaxel Resistant Prostate Cancer Cells have Potent Tumor Initiating Capacity

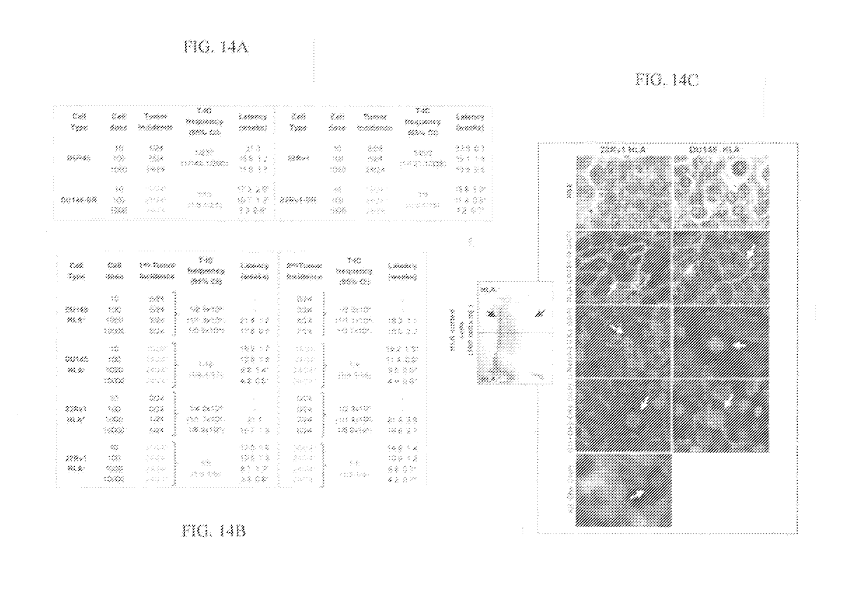

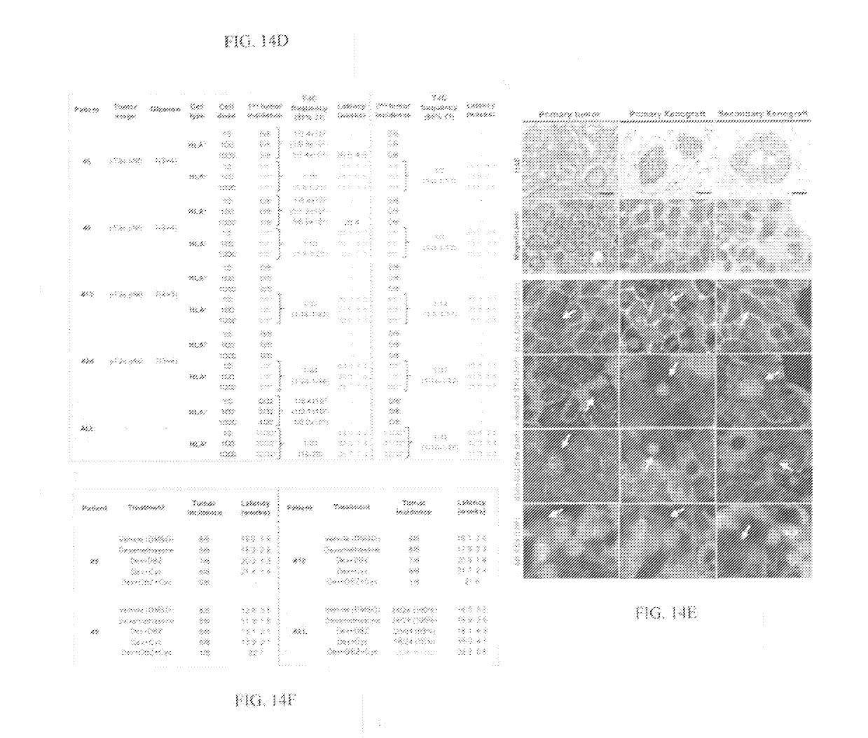

In recent years, a number of studies have shown that tumor initiating cells (T-ICs) may preferentially survive exposure to chemotherapy, providing an attractive rationale for relapse following initial tumor shrinkage with standard therapy (Corbin et al., 2011; Ishikawa et al., 2007; Lonardo et al., 2011; Todaro et al., 2007; Yu et al., 2007). Having demonstrated that CK-negative cells survive Docetaxel exposure in vitro and in vivo, experiments were performed to investigate the tumor initiating capacity of these cells. Since efficient xenotransplantation is a major criterion for the validation of a T-IC enriched compartment (Dalerba et al., 2007; Vermeulen et al., 2008; Visvader and Lindeman, 2008), serial dilution tumor initiation assays were performed using the Docetaxel resistant models. Interestingly, DU145-DR and 22Rv1-DR cells had higher tumor initiating capacity than their parental sensitive cells when injected into NOD/SCID IL-2 receptor gamma chain null (NSG) mice (FIG. 14A). In DU145-DR cell lines, there was one T-IC in 15 cells (95% CI: 9 to 27) versus one T-IC in 237 cells (95% CI: 144 to 5390) in DU145 parental cells. In 22Rv1-DR cell lines, there was one T-IC in 9 cells (95% CI: 5 to 15) versus one T-IC in 202 cells (95% CI: 121 to 338) in the 22Rv1 parental cells. These results indicate that DU145 and 22Rv1 Docetaxel resistant cells display 15.8- and 22.4-fold higher tumor initiating capacity than their parental cells, respectively.

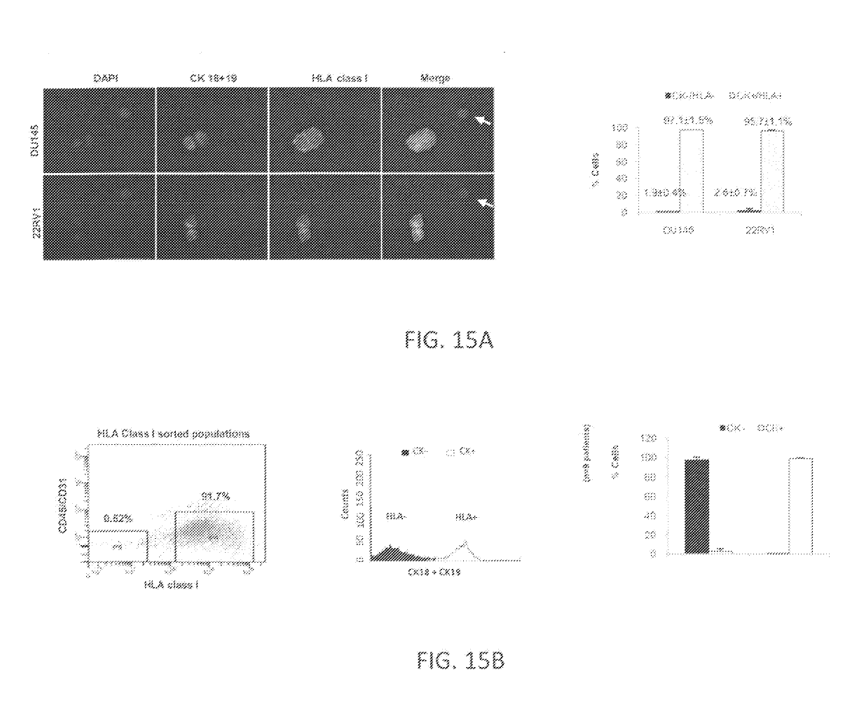

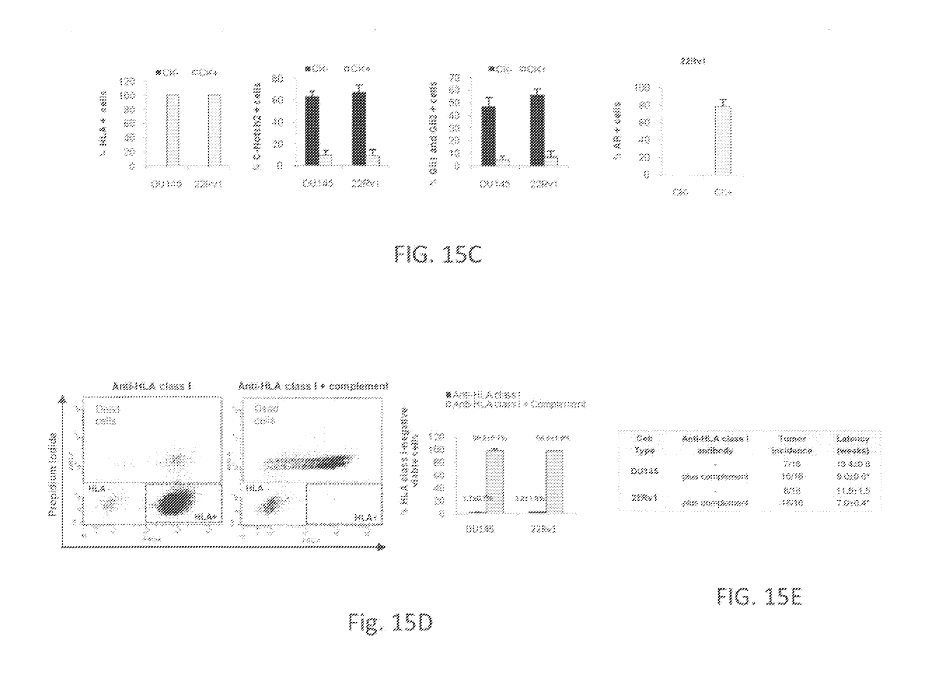

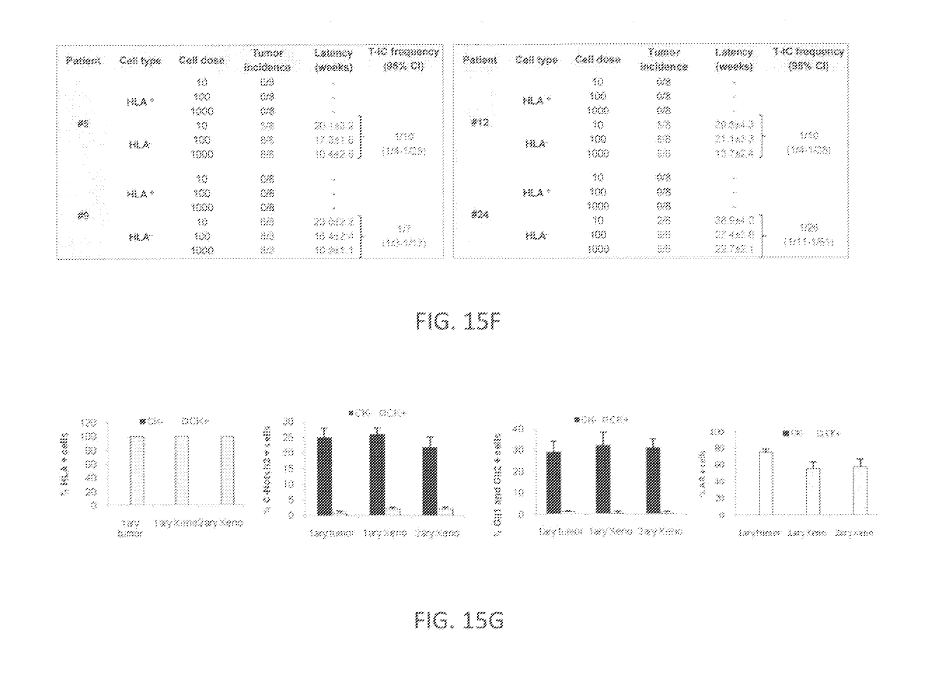

One feature of the Docetaxel resistance phenotype was lack of HLA class I expression (FIGS. 2 and 3). Therefore, it was reasoned that viable CK-negative Docetaxel resistant cells could be isolated from bulk populations using HLA class I expression as a cell surface marker. Indeed, double marker flow cytometry analysis revealed that HLA class I negativity closely overlapped with the CK-negative population both in cell lines (FIG. 15A) and in primary prostate cancers (FIG. 15B). Thus, viable CK-negative and CK-positive subpopulations could be isolated by flow cytometry using HLA class I expression.

Next, HLA class I was used as a cell surface marker to perform limiting dilution tumor initiation assays in NSG mice. In DU145 cell lines, there was one T-IC in 10 cells (95% CI: 6 to 17) in the HLA class I-negative compartment versus one T-IC in 2.5×10⁴ cells (95% CI: 1.3×10⁴ to 5.0×10⁴) in the HLA class I-positive compartment (FIG. 14B). In 22Rv1 cell lines, there was one T-IC in 5 cells (95% CI: 3 to 8) in the HLA class I-negative compartment versus one T-IC in 4.0×10⁴ cells (95% CI: 1.7×10⁴ to 8.9×10⁴) in the HLA class I-positive compartment. Furthermore, primary xenografts formed from HLA class I-negative/CK-negative cells recapitulated the phenotypic heterogeneity of the parental cell lines with an HLA class I-positive/CK-positive phenotype in the majority of the tumor cells, as well as a small HLA class I-negative/CK-negative population that expressed nuclear cleaved Notch2, Gli1, and Gli2, and lacked nuclear AR (FIGS. 14C and 15C). Further, HLA class I-negative cells isolated from primary xenografts serially engrafted into secondary recipients, whereas HLA class I-positive cells rarely engrafted (FIG. 14B). Thus, in both DU145 and 22Rv1 cell lines, the tumor initiating capacity of HLA class I-negative cells was ˜2,000-fold higher than HLA class I-positive cells. Moreover, complement-mediated lysis was used as an alternative method to show the presence of an HLA class I-negative cell compartment with high tumor initiating capacity in the parental cells. Incubation of DU145 and 22Rv1 parental cells with HLA class I antibody and complement induced a robust depletion of HLA class I-positive cells, whereas HLA class I-negative cells remained viable (FIG. 15D). The surviving HLA class I-negative population exhibited robust tumor initiating capacity in comparison to the non-complement depleted cells (FIG. 15E).

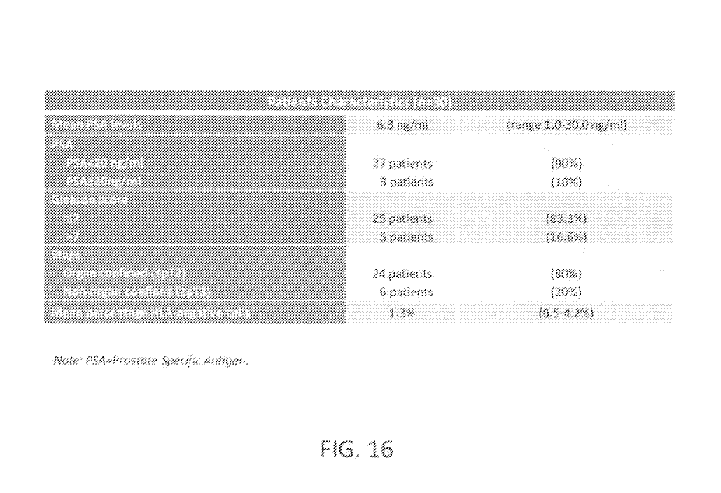

To investigate the tumorigenic capacity of the identified prostate T-IC population in fresh tumors, primary prostate cancer tissue samples were used for analysis. The presence of adenocarcinoma was confirmed histologically in the processed tissue in 30 patients (FIG. 16). Overall, the injection of cells from 4 out of the 30 (13.3%) confirmed individual prostate cancer samples generated tumor xenografts after a median follow-up time of 55.6 weeks (range 37.3-62.0). Among these 4 patient samples, the HLA class I-negative cells displayed higher tumorigenic potential compared to the HLA class I-positive cells (FIG. 14D). After 8 injections for each serial dilution in NOD/SCID mice, HLA class I-positive cells only generated tumors at the highest injection dose, and with poor frequency (12.5%). On the contrary, tumors were consistently generated after injection of 1,000 HLA class I-negative cells (100%), and injection of as few as 10 HLA class I-negative cells resulted in tumor growth (46.8%). There was one T-IC in 25 (95% CI: 16 to 39) HLA class I-negative cells versus one T-IC in 8.4×10³ (95% CI: 3.1×10³ to 2.2×10⁴) HLA class I-positive cells. Thus, the tumor initiating capacity was 336-fold higher in HLA class I-negative cells. Moreover, in a subanalysis using HLA class I sorted cells from primary xenografts injected into NSG mice, HLA class I-negative cells continued to form tumors efficiently, while HLA class I-positive cells failed to engraft, indicating that tumor initiation of HLA class I-negative cells was independent of a remnant host immune response (FIG. 15F). Immunohistochemistry of tumors derived from HLA class I-negative cells showed that they faithfully reproduced the phenotype of the primary prostate tumor with the expression of epithelial and prostate related markers (CKs and AR) as well as HLA class I antigens in the majority of tumor cells, as well as a small HLA class I-negative/CK-negative compartment characterized by lack of AR and expression of nuclear cleaved Notch2, Gli1, and Gli2 (FIGS. 14E and 15G). Due to the long latency period of the generated prostate cancer xenografts, their identity was confirmed by short tandem repeat DNA fingerprinting (data not shown). Further, HLA class I-negative cells isolated from primary xenografts serially engrafted into secondary recipients, whereas HLA class I-positive cells failed to engraft (FIG. 14D). Taken together, these results suggest that the HLA class I-negative subpopulation is highly enriched in T-ICs that sustain serial xenotransplantation and reproduce the phenotypic heterogeneity of the primary tumor.

Finally, given that the data in the previous examples suggested that Notch and Hedgehog signaling were required to sustain the viability of CK-negative/HLA class I-negative cells, experiments were performed to assess whether inhibition of these pathways could reduce the tumor initiating capacity of these cells. 100 HLA class I-negative sorted cells from human prostate cancer xenografts #5, #9, and #12 were injected subcutaneously into NSG mice and treated with vehicle solution, Dexamethasone alone, dual drug combinations (e.g. Dexamethasone plus Cyclopamine), or triple drug combination (Dexamethasone plus Cyclopamine and DBZ). Mice treated with the combination of Notch and Hedgehog inhibitors showed a significant (p<0.0001) reduction in tumor incidence when compared to mice treated with vehicle solution (DMSO) or each inhibitor alone (FIG. 14F).

### Other Embodiments

While the present technology has been described in conjunction with the detailed description thereof, the foregoing description is intended to illustrate and not limit the scope of the technology. Other aspects, advantages, and modifications are contemplated herein, as set forth in the following claims.

We claim:

1. A method of preventing or reducing resistance to a first chemotherapy agent in a cancer, the method comprising administering to a subject having the cancer one or both of a Notch signaling pathway inhibitor or a Hedgehog signaling pathway inhibitor.

2. The method of claim 1, further comprising administering a further chemotherapy agent to the subject, the further chemotherapy agent being the same or different from the first chemotherapy agent.

3. The method of claim 2, wherein the further chemotherapy agent is administered prior to, after, or concurrently with, the Notch signaling pathway inhibitor or Hedgehog signaling pathway inhibitor.

4. The method of claim 1, wherein the first chemotherapy agent is Docetaxel.

5. The method of claim 1, wherein the cancer is prostate cancer.

6. The method of claim 1, wherein the Notch signaling pathway inhibitor is a Notch antibody, a nucleic acid that inhibits Notch activity, DBZ, Compound E, or a PI3K/AKT pathway inhibitor.

7. The method of claim 6, wherein the PI3K/AKT pathway inhibitor is LY294002.

8. The method of claim 6, wherein the nucleic acid is a short hairpin RNA or a nucleic acid that complementary to a Notch nucleic acid or fragment thereof.

9. The method of claim 1, wherein the Hedgehog signaling pathway inhibitor is a Hedgehog antibody, a nucleic acid that inhibits Hedgehog activity Cyclopamine, GDC-0449, a Bcl-2 family member inhibitor, or a short hairpin RNA that targets Gli1 or Gli2.

10. The method of claim 9, wherein the Bcl-2 family member inhibitor is ABT-737.

11. A method of treating cancer, the method comprising administering to a subject having the cancer one or both of a Notch signaling pathway inhibitor or a Hedgehog signaling pathway inhibitor.

12. A method of identifying a tumor cell that is resistant to a chemotherapy agent, the method comprising detecting activation of the Notch or Hedgehog signaling pathways, wherein the activation indicates the resistant tumor cell.

13. A method of predicting the predicting survival of a subject having cancer, the method comprising detecting activation of the Notch or Hedgehog signaling pathways, wherein said activation indicates a decreased survival time.

14. The method of claim 13, wherein the activation further indicates tumor aggressiveness and poor patient prognosis.

15. The method of claim 13, wherein the subject has previously received treatment for the cancer.

16. The method of claim 15, wherein the treatment comprises administration of a chemotherapy agent.

17. The method of claim 16, wherein the treatment comprises Docetaxel.

18. The method of claim 13, wherein the detecting activation comprises detection of one or more of:
a) cleaved Notch2;
b) increased expression of Gli1;
c) increased expression of Gli2;
d) reduced expression of Patched;
d) phosphorylation of AKT (Ser473); or
e) increased levels of Bcl-2.

19. A method of identifying a tumor cell resistant to a chemotherapy agent, the method comprising detecting decreased expression of an HLA class I antigens, a cytokeratin 18, a cytokeratins 19 or any combination thereof compared to a normal control cell, wherein the decreased expression indicates a tumor cell that is resistant to the chemotherapy agent.

20. The method of claim 19, wherein the chemotherapy agent comprises Docetaxel.


SEQUENCE LISTING (38 sequences)

SEQ ID NO: 1 - DNA, 21 bp, Artificial Sequence
  Description of Artificial Sequence Synthetic primer
aacgcatgctttggggggatg

SEQ ID NO: 2 - DNA, 23 bp, Artificial Sequence
  Description of Artificial Sequence Synthetic primer
acaaacatcacagaaacgacagt

SEQ ID NO: 3 - DNA, 21 bp, Artificial Sequence
  Description of Artificial Sequence Synthetic primer
tgacaggctaggcttcaaggt

SEQ ID NO: 4 - DNA, 23 bp, Artificial Sequence
  Description of Artificial Sequence Synthetic primer
atcgcattatgcaagactggatt

SEQ ID NO: 5 - DNA, 23 bp, Artificial Sequence
  Description of Artificial Sequence Synthetic primer
atgcacatccttcgataagactg

SEQ ID NO: 6 - DNA, 22 bp, Artificial Sequence
  Description of Artificial Sequence Synthetic primer
ttggaaccagatgcacattgat

SEQ ID NO: 7 - DNA, 20 bp, Artificial Sequence
  Description of Artificial Sequence Synthetic primer
tcttgcgactcacgcttgac

SEQ ID NO: 8 - DNA, 21 bp, Artificial Sequence
  Description of Artificial Sequence Synthetic primer
tgttcaaacttctgctcctga

SEQ ID NO: 9 - DNA, 20 bp, Artificial Sequence
  Description of Artificial Sequence Synthetic primer
cccatcattgcaatagcagg

SEQ ID NO: 10 - DNA, 23 bp, Artificial Sequence
  Description of Artificial Sequence Synthetic primer
gagacgtacagtccagtccttgg

SEQ ID NO: 11 - DNA, 19 bp, Artificial Sequence
  Description of Artificial Sequence Synthetic primer
ccacctccctcaggctgtt

SEQ ID NO: 12 - DNA, 21 bp, Artificial Sequence
  Description of Artificial Sequence Synthetic primer
ctgcgggacaagattcttggt

SEQ ID NO: 13 - DNA, 19 bp, Artificial Sequence
  Description of Artificial Sequence Synthetic primer
ccagacgggcattgtcgat

SEQ ID NO: 14 - DNA, 19 bp, Artificial Sequence
  Description of Artificial Sequence Synthetic primer
cccaactccacaggcatac

SEQ ID NO: 15 - DNA, 20 bp, Artificial Sequence
  Description of Artificial Sequence Synthetic primer
acacgaactccttccgctcc

SEQ ID NO: 16 - DNA, 20 bp, Artificial Sequence
  Description of Artificial Sequence Synthetic primer
acaccaaccagaacaagcag

SEQ ID NO: 17 - DNA, 20 bp, Artificial Sequence
  Description of Artificial Sequence Synthetic primer
agtcttcccagtggcagttg

SEQ ID NO: 18 - DNA, 23 bp, Artificial Sequence
  Description of Artificial Sequence Synthetic primer
ctggaaatgacagtgaagcacct

SEQ ID NO: 19 - DNA, 22 bp, Artificial Sequence
  Description of Artificial Sequence Synthetic primer
attgatctgggtcatgcagttg

SEQ ID NO: 20 - DNA, 22 bp, Artificial Sequence
  Description of Artificial Sequence Synthetic primer
gctggtacccagtgcttttgag

SEQ ID NO: 21 - DNA, 21 bp, Artificial Sequence
  Description of Artificial Sequence Synthetic primer
tgcaggatctcggctttttct

SEQ ID NO: 22 - DNA, 23 bp, Artificial Sequence
  Description of Artificial Sequence Synthetic primer
aaaaggagggagttacactcagg

SEQ ID NO: 23 - DNA, 21 bp, Artificial Sequence
  Description of Artificial Sequence Synthetic primer
gctgtgagggacacatcagag

SEQ ID NO: 24 - DNA, 20 bp, Artificial Sequence
  Description of Artificial Sequence Synthetic primer
cagttcgtgaggttcgacag

SEQ ID NO: 25 - DNA, 19 bp, Artificial Sequence
  Description of Artificial Sequence Synthetic primer
cagccgtacatgctctgga

SEQ ID NO: 26 - DNA, 25 bp, Artificial Sequence
  Description of Artificial Sequence Synthetic primer
ggcattaatcgctacagttgtgtct

SEQ ID NO: 27 - DNA, 22 bp, Artificial Sequence
  Description of Artificial Sequence Synthetic primer
ggaggcacactcatcaatgtca

SEQ ID NO: 28 - DNA, 22 bp, Artificial Sequence
  Description of Artificial Sequence Synthetic primer
ttgcttgggagtcattaactgg

SEQ ID NO: 29 - DNA, 22 bp, Artificial Sequence
  Description of Artificial Sequence Synthetic primer
cccacaatcaactcctcctgcc

SEQ ID NO: 30 - DNA, 21 bp, Artificial Sequence
  Description of Artificial Sequence Synthetic primer
atggatggtgcccgccgagag

SEQ ID NO: 31 - DNA, 22 bp, Artificial Sequence
  Description of Artificial Sequence Synthetic primer
atggtctcgttgatcttgctgg

SEQ ID NO: 32 - DNA, 20 bp, Artificial Sequence
  Description of Artificial Sequence Synthetic primer
aaactggaacggtgaaggtg

SEQ ID NO: 33 - DNA, 20 bp, Artificial Sequence
  Description of Artificial Sequence Synthetic primer
gtggcttttaggatggcaag

SEQ ID NO: 34 - DNA, 39 bp, Artificial Sequence
  Description of Artificial Sequence Synthetic primer
aaaaagaattccgccaccatggcgcacgctgggagaaca

SEQ ID NO: 35 - DNA, 33 bp, Artificial Sequence
  Description of Artificial Sequence Synthetic primer
aaaagcggccgctcacttgtggcccagataggc

SEQ ID NO: 36 - DNA, 22 bp, Artificial Sequence
  Description of Artificial Sequence Synthetic primer
tccccctttactcggcccccac

SEQ ID NO: 37 - DNA, 22 bp, Artificial Sequence
  Description of Artificial Sequence Synthetic primer
ttcctgcgttatcccctgattc

SEQ ID NO: 38 - DNA, 24 bp, Artificial Sequence
  Description of Artificial Sequence Synthetic primer
gctcctccggcccttgctcaccat